(19) United States
(12) Patent Application Publication
(10) Pub. No.: US 2023/0301172 A1
(43) Pub. Date: Sep. 21, 2023

(54) THERMALLY ACTIVATED DELAYED FLUORESCENT MATERIAL, ORGANIC LIGHT-EMITTING DEVICE AND DISPLAY APPARATUS

(71) Applicant: BOE Technology Group Co., Ltd., Beijing (CN)

(72) Inventors: Xinghua LIU, Beijing (CN); Xiaojin ZHANG, Beijing (CN); Haiyan SUN, Beijing (CN)

(21) Appl. No.: 18/021,548

(22) PCT Filed: Mar. 3, 2022
(86) PCT No.: PCT/CN2022/079125

§ 371 Date: Feb. 15, 2023

(30) Foreign Application Priority Data
Jun. 30, 2021 (CN) 202110732384.6

(51) Int. Cl. H10K 85/40; H10K 85/60; C09K 11/02; C09K 11/06; H10K 50/15; H10K 50/18; H10K 50/17

(52) U.S. Cl. CPC H10K 85/40; H10K 85/654; H10K 85/6572; H10K 85/633; H10K 85/658; C09K 11/02; C09K 11/06; H10K 85/615; H10K 85/6574; H10K 50/157; H10K 50/181; H10K 50/171; H10K 2101/20

(57) ABSTRACT

Provided in the present disclosure are a thermally activated delayed fluorescent material, an organic light-emitting device and a display apparatus. The energy level difference between a singlet energy level and a triplet energy level of the thermally activated delayed fluorescent material is less than 0.3 eV, and a spin-orbit coupling value SOC between a singlet state and a triplet state of the thermally activated delayed fluorescent material is SOC≤0.05 cm⁻¹.

### CROSS REFERENCE TO RELATED APPLICATIONS

This application is a US National Stage of International Application No. PCT/CN2022/079125, filed on Mar. 3, 2022, which claims the priority from Chinese patent application No. 202110732384.6, filed with the China National Intellectual Property Administration on Jun. 30, 2021 and entitled “THERMALLY ACTIVATED DELAYED FLUORESCENCE MATERIAL, ORGANIC LIGHT-EMITTING DEVICE AND DISPLAY APPARATUS”, the entire contents of which are incorporated herein by reference.

### FIELD

The disclosure relates to the technical field of display, in particular to a thermally activated delayed fluorescence material, an organic light-emitting device and a display apparatus.

### BACKGROUND

A thermally activated delayed fluorescence (TADF) technology, as an organic light-emitting diode technology with application potential, has achieved rapid development in recent years, and is known as a third generation organic light-emitting diode (OLED) technology. A superfluorescence technology based on a TADF sensitizer is considered to be the most valuable TADF implementation, and has great potential for application in the next-generation flat panel displays, thus becoming a hotspot for research and development.

However, at present, the superfluorescence technology also faces many problems, such as low device efficiency and short lifetime, which prevent its practical application.

### SUMMARY

An embodiment of the disclosure provides a thermally activated delayed fluorescence material, an organic light-emitting device and a display apparatus, so as to solve one or more of the problems existing in the prior art.

Therefore, an embodiment of the disclosure provides a thermally activated delayed fluorescence material. The energy gap between a singlet state and a triplet state of the thermally activated delayed fluorescence material is less than 0.3 eV, and a spin-orbit coupling (SOC) value between the singlet state and the triplet state of the thermally activated delayed fluorescence material is greater than or equal to 0.05 cm⁻¹.

In some embodiments, in the above thermally activated delayed fluorescence material provided by the embodiment of the disclosure, the thermally activated delayed fluorescence material has a structure represented by the following formula (1):

D-Ln-A (1)
in the formula (1), D is a donor group, L is a linking group, and A is an acceptor group;
where D is selected from at least one of carbazolyl, arylamino, alkylamino, silyl, alkoxy, aryloxy, thio, alkylthio, arylthio, acridinyl, phenoxazine, or phenothiazine;
L is selected from at least one of a single bond, —O—, phenyl, biphenyl, cycloalkylene, arylene, heteroaryl, heterocycloalkylene, or heteroalkenylene, and n is 1-4; and
A is selected from at least one of fluorine, cyano, triazine, cyanobenzene, pyridine, phosphinoxy, ketocarbonyl, sulfonyl, pyrrolyl, thienyl, pyrazolyl, thiazolyl, pyridyl, pyrazinyl, pyrimidinyl, pyridazinyl, or phenalenylene.

In some embodiments, in the above thermally activated delayed fluorescence material provided by the embodiment of the disclosure, the formula (1) is selected from the following compounds:

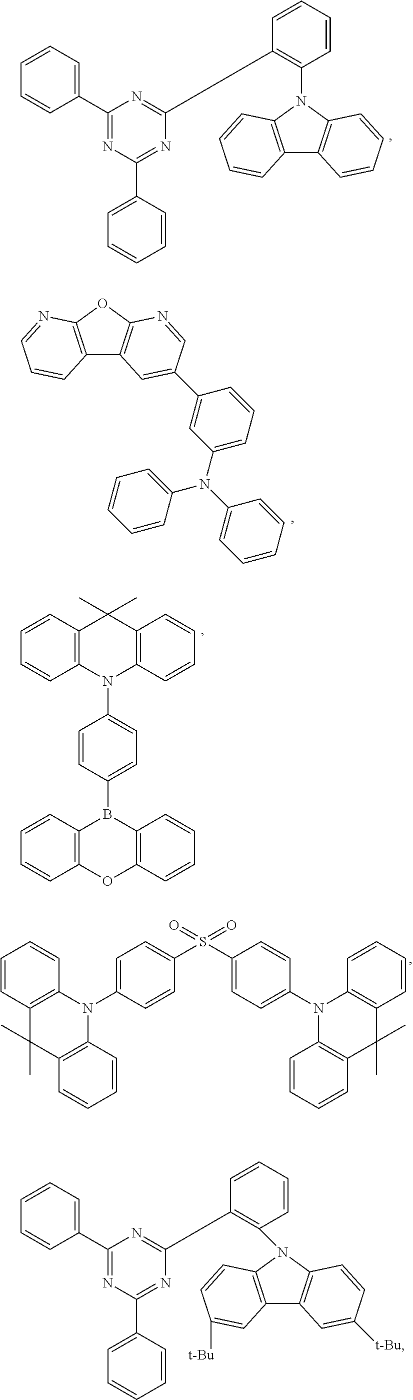

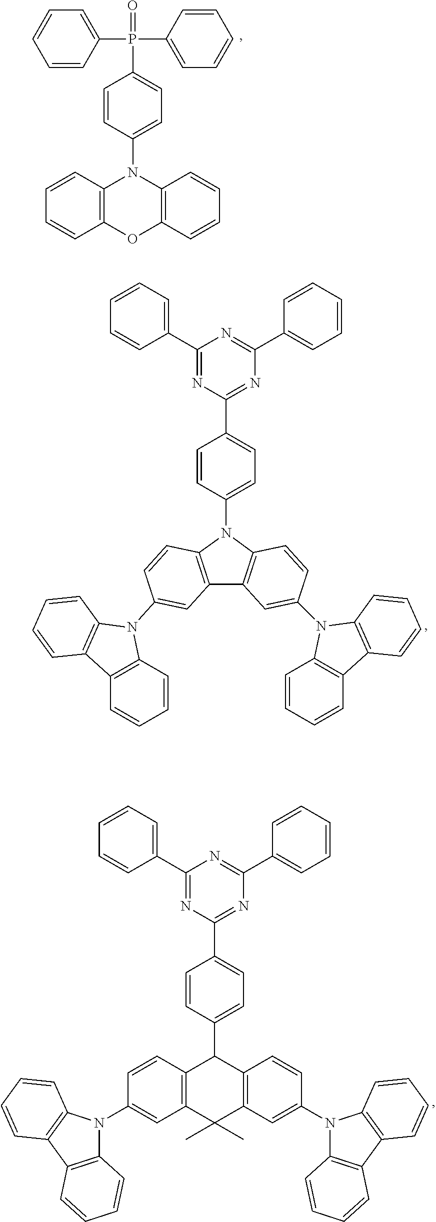

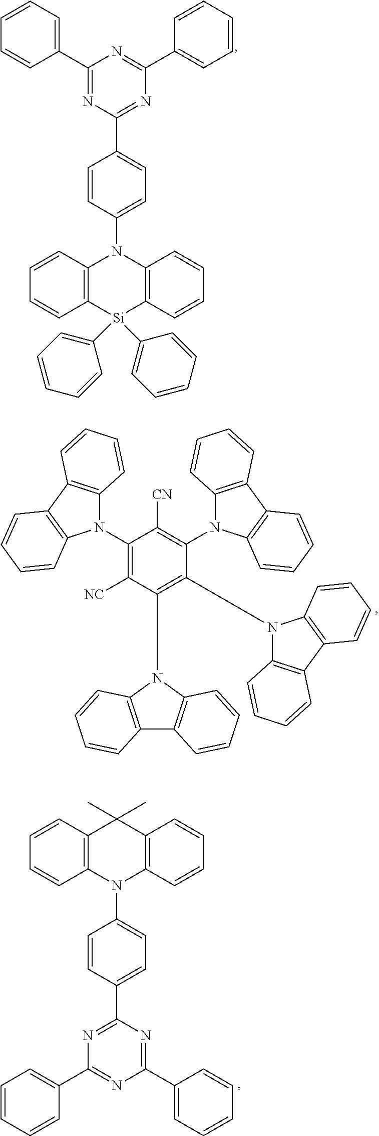

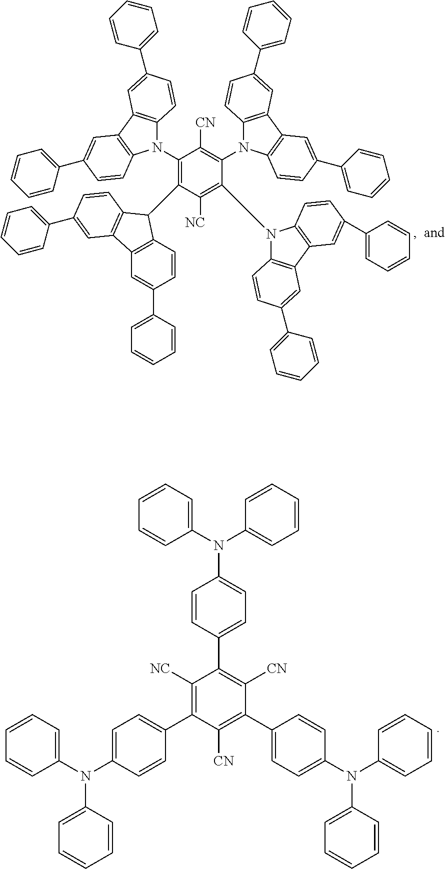 [SMILES: N#Cc1c(C2c3ccc(-c4ccccc4)cc3-c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-c2ccc(N(c3ccccc3)c3ccccc3)cc2)c(C#N)c(-c2ccc(N(c3ccccc3)c3ccccc3)cc2)c(C#N)c1-c1ccc(N(c2ccccc2)c2ccccc2)cc1]

Accordingly, an embodiment of the disclosure also provides an organic light-emitting device, including:
an anode layer;
a cathode layer, disposed opposite to the anode layer; and
a light-emitting layer, disposed between the anode layer and the cathode layer, where the light-emitting layer includes a host material, a guest material, and the above thermally activated delayed fluorescence material provided by the embodiment of the disclosure.

In some embodiments, the above organic light-emitting device provided by the embodiment of the disclosure further includes a hole-blocking layer between the light-emitting layer and the cathode layer, where a material of the hole-blocking layer has a structure represented by the following formula (2):

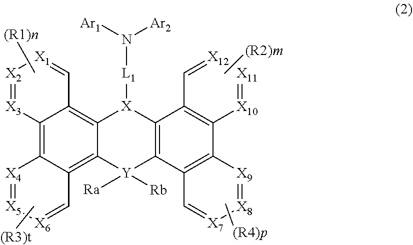

in the formula (2), at least one of X₁ to X₁₂ is N;
X is B or N, Y is C or Si, and n, m, t, and p are each independently an integer of 0-4;
R1 to R4 are each independently substituted or unsubstituted C6-60 aryl, or R1 to R4 are each independently substituted or unsubstituted C2-60 heteroaryl containing heteroatoms selected from any one or more of N, O and S;
L₁ is a single bond, or L₁ is substituted or unsubstituted C6-60 arylene, or L₁ is substituted or unsubstituted C2-60 heteroarylene containing heteroatoms selected from any one or more of N, O, and S; and
Ar₁ and Ar₂ are each independently substituted or unsubstituted C6-60 aryl, or Ar₁ and Ar₂ are each independently substituted or unsubstituted C2-60 heteroaryl containing heteroatoms selected from any one or more of N, O, and S.

In some embodiments, in the above organic light-emitting device provided by the embodiment of the disclosure, the formula (2) is selected from the following compounds:

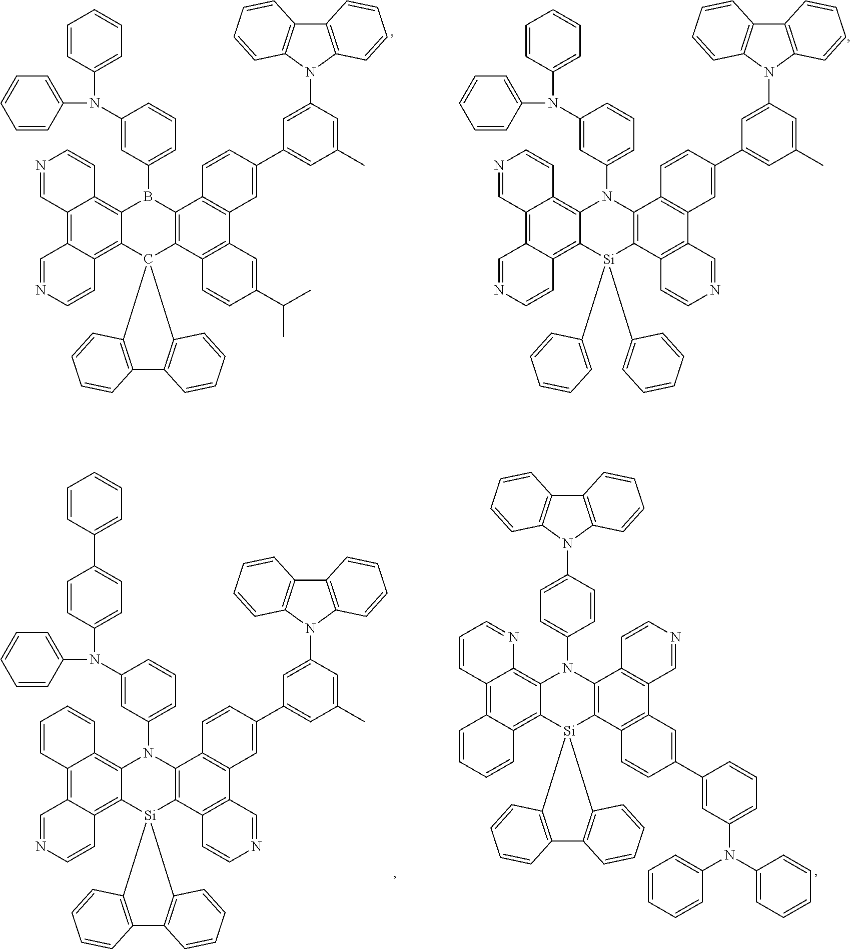

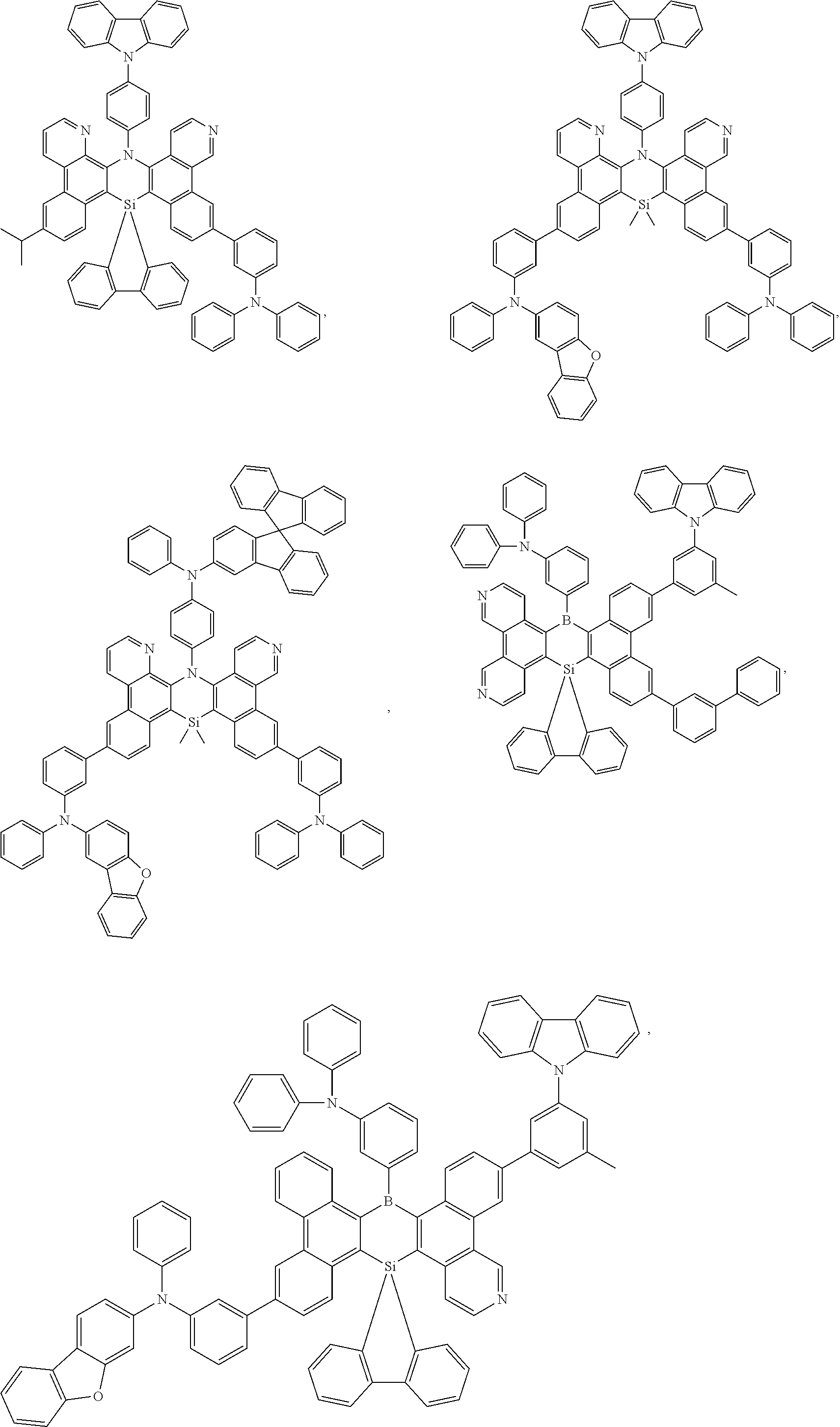

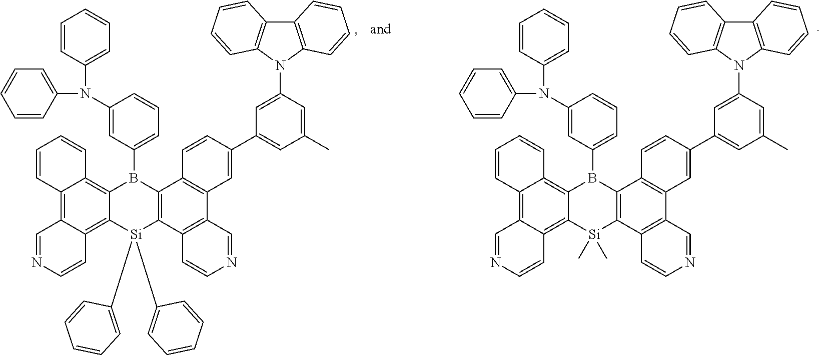 [SMILES: Cc1cc(-c2ccc3c4c(c5ccncc5c3c2)[Si](c2ccccc2)(c2ccccc2)c2c(c3ccccc3c3cnccc23)B4c2cccc(N(c3ccccc3)c3ccccc3)c2)cc(-n2c3ccccc3c3ccccc32)c1 ; Cc1cc(-c2ccc3c4c(c5ccncc5c3c2)[Si](C)(C)c2c(c3ccccc3c3cnccc23)B4c2cccc(N(c3ccccc3)c3ccccc3)c2)cc(-n2c3ccccc3c3ccccc32)c1]

In some embodiments, in the above organic light-emitting device provided by the embodiment of the disclosure, the lowest triplet energy of the host material in the light-emitting layer is lower than the lowest triplet energy of the material of the hole-blocking layer.

In some embodiments, in the above organic light-emitting device provided by the embodiment of the disclosure, the HOMO energy level of the host material in the light-emitting layer is lower than the HOMO energy level of the material of the hole-blocking layer.

In some embodiments, in the above organic light-emitting device provided by the embodiment of the disclosure, the lowest triplet energy of the thermally activated delayed fluorescence material in the light-emitting layer is lower than the lowest triplet energy of the material of the hole-blocking layer.

In some embodiments, in the above organic light-emitting device provided by the embodiment of the disclosure, the HOMO energy level of the thermally activated delayed fluorescence material in the light-emitting layer is less than the HOMO energy level of the material of the hole-blocking layer.

In some embodiments, the above organic light-emitting device provided by the embodiment of the disclosure further includes an electron transport layer between the hole-blocking layer and the cathode layer, an electron injection layer between the electron transport layer and the cathode layer, an electron-blocking layer between the light-emitting layer and the anode layer, a hole transport layer between the electron-blocking layer and the anode layer, and a hole injection layer between the hole transport layer and the anode layer.

In some embodiments, in the above organic light-emitting device provided by the embodiment of the disclosure, the guest material is a fluorescent material or a phosphorescent material.

In some embodiments, in the above organic light-emitting device provided by the embodiment of the disclosure, a material of the anode layer is ITO, a material of the hole injection layer is

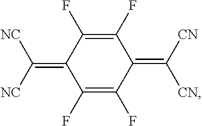 [SMILES: N#CC(C#N)=c1c(F)c(F)c(=C(C#N)C#N)c(F)c1F]

a material of the hole transport layer is

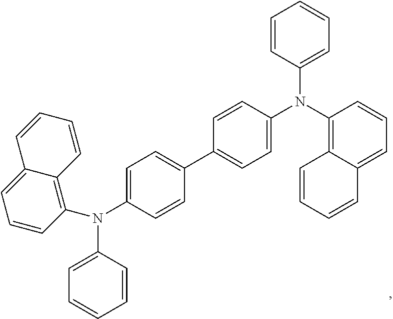 [SMILES: c1ccc(N(c2ccc(-c3ccc(N(c4ccccc4)c4cccc5ccccc45)cc3)cc2)c2cccc3ccccc23)cc1]

a material of the electron-blocking layer is

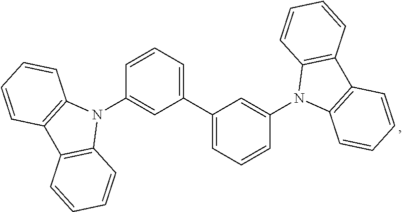 [SMILES: c1cc(-c2cccc(-n3c4ccccc4c4ccccc43)c2)cc(-n2c3ccccc3c3ccccc32)c1]

the host material is

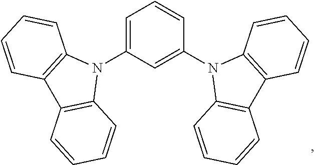 [SMILES: c1cc(-n2c3ccccc3c3ccccc32)cc(-n2c3ccccc3c3ccccc32)c1]

the thermally activated delayed fluorescence material is

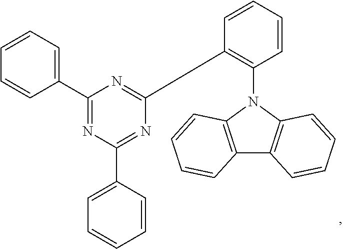 [SMILES: c1ccc(-c2nc(-c3ccccc3)nc(-c3ccccc3-n3c4ccccc4c4ccccc43)n2)cc1]

the guest material is

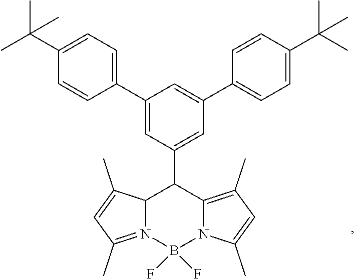

a material of the hole-blocking layer is

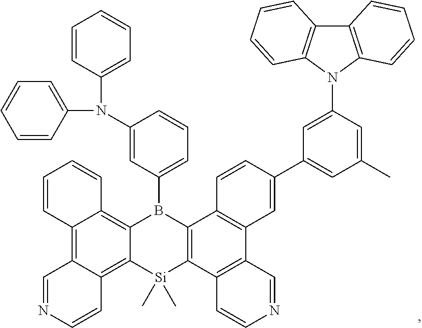 [SMILES: Cc1cc(-c2ccc3c4c(c5ccncc5c3c2)[Si](C)(C)c2c(c3ccccc3c3cnccc23)B4c2cccc(N(c3ccccc3)c3ccccc3)c2)cc(-n2c3ccccc3c3ccccc32)c1]

a material of the electron transport layer is

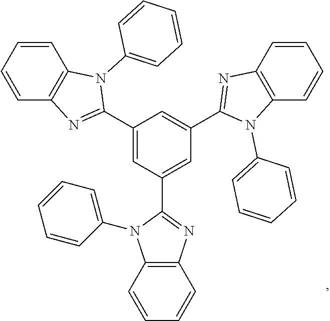 [SMILES: c1ccc(-n2c(-c3cc(-c4nc5ccccc5n4-c4ccccc4)cc(-c4nc5ccccc5n4-c4ccccc4)c3)nc3ccccc32)cc1]

a material of the electron injection layer is

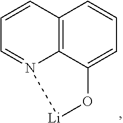

and a material of the cathode layer is a Mg/Ag alloy.

Accordingly, an embodiment of the disclosure also provides a display apparatus, including any one of the organic light-emitting devices described above.

### BRIEF DESCRIPTION OF THE DRAWINGS

FIG. 1 is a schematic structural diagram of an organic light-emitting device according to an embodiment of the disclosure.

FIG. 2 shows a hydrogen nuclear magnetic resonance spectrum (1H-NMR) of a formula 2-10 according to an embodiment of the disclosure.

FIG. 3 shows a carbon nuclear magnetic resonance spectrum (13C-NMR) in the formula 2-10 according to an embodiment of the disclosure.

FIG. 4 shows a hydrogen nuclear magnetic resonance spectrum (1H-NMR) of a formula 2-11 according to an embodiment of the disclosure.

FIG. 5 shows a carbon nuclear magnetic resonance spectrum (13C-NMR) of the formula 2-11 according to an embodiment of the disclosure.

### DETAILED DESCRIPTION

In order to make the objective, technical solutions and advantages of the disclosure clearer, the disclosure will be further described in detail below with reference to the accompanying drawings. Obviously, the described embodiments are only part of the embodiments of the disclosure, but not all the embodiments. Based on the embodiments of the disclosure, other embodiments obtained by those of ordinary skill in the art without creative work all belong to the scope of protection of the disclosure.

Shapes and dimensions of components in the accompanying drawings do not reflect a real scale, and are only intended to illustrate the disclosure.

In a Thermally Activated Delayed Fluorescence (TADF) mechanism, for the organic molecule having smaller singlet—triplet (S1-T1) energy gaps (ΔE_{ST}), the small energy gap enables reverse intersystem crossing (RISC) to occur, where excitons in the T₁ are converted to S₁ in a thermally activated process.

In 2014, the professor Adachi proposed a hyperfluorescence light-emitting technology using a ternary light-emitting layer system, i.e. a wide-bandgap host (a host material), TADF as a sensitizer, and a fluorescent emitter (a guest material). Excitons are generated from the TADF sensitizer, 25% of the excitons enter a singlet state and 75% of the excitons enter a triplet state. For the TADF materials, excitons in triplet state are converted to a singlet state by RISC, and then the singlet energy is transferred from the TADF material to the fluorescent emitter by Förster resonance energy transfer (FET), resulting in exciton recombination in the TADF materials and light emission from the fluorescent emitter, thus achieving an internal quantum efficiency (IQE) of 100%.

To reduce the device voltage and improve the device efficiency by using the reverse intersystem crossing characteristics of the TADF materials, it is necessary for the TADF materials to have a faster RISC rate, and the RISC rate is directly proportional to a S1-T1 spin-orbit coupling (SOC) value, and is inversely proportional to the S1-T1 energy gaps (ΔE_{ST}).

One electron is removed from the HOMO energy level of one donor molecule to the LUMO level of one acceptor molecule, and an interrelated electron-hole pair, i.e., an exciton is formed, where an electron acceptor can be a nearby molecule or a molecule with a certain distance. The binding energy between the electron-hole pair causes the energy level with the transferred electron on the acceptor molecule to decrease compared to the situation without accepting the electron, forming a charge transfer state. The energy level of the charge transfer state is related to the position, and can be divided into the energy level of a triplet charge transfer state and the energy level of a singlet charge transfer state, the TADF property is related to the degree of overlap of the HOMO level and LUMO level of the donor, and when the orbital overlap degree increases, the energy level difference ΔE_{ST} becomes larger, and even larger than 0.3 eV, and the material will lose the TADF property. The TADF requires ΔE_{ST}<0.3 ev, and the greater the distance between the electron-hole pair, the smaller their correlation degree, which can reduce the S1-T1 energy gaps.

The magnitude of the energy level difference ΔE_{ST} only determines whether the material has the TADF property, while the S1-T1 spin-orbit coupling (SOC) value of the material determines the RISC rate.

Based on this, an embodiment of the disclosure provides a thermally activated delayed fluorescence material. The energy level difference between a singlet energy level and a triplet energy level of the thermally activated delayed fluorescence material is less than 0.3 eV, and a spin-orbit coupling (SOC) value between a singlet state and a triplet state of the thermally activated delayed fluorescence material is greater than or equal to 0.05 cm⁻¹.

According to the above thermally activated delayed fluorescence material provided by the embodiment of the disclosure, a thermally activated delayed fluorescence material having both a large SOC value (≥0.05 cm-1) and small ΔEST (less than 0.3 eV) is used, which is beneficial to increase the rate of conversion of triplet (T1) excitons to singlet (S1) excitons of the thermally activated delayed fluorescence material, the triplet excitons in the thermally activated delayed fluorescence material can be more easily converted to the singlet state to form singlet excitons via reverse intersystem crossing. When the thermally activated delayed fluorescence material is applied to a light-emitting layer of an organic light-emitting device, the degradation of the device performance due to the annihilation of triplet excitons can be greatly reduced.

It should be noted that the SOC value, expressed as <Ψ_{S1}|Ĥ_{SO}|Ψ_{T1}> by using quantum mechanical symbols, where Ĥ_{SO} is an operator of SOC, is calculated based on the configuration of S1 by simulation calculation using a time-dependent density functional theory (TDDFT), and by using the M062X/6-31G (d,p) level.

In some embodiments, in the above thermally activated delayed fluorescence material provided by the embodiment of the disclosure, the thermally activated delayed fluorescence material has a structure represented by the following formula (1):

D-Ln-A (1)
in the formula (1), D is a donor group, L is a linking group, and A is an acceptor group; where D is selected from at least one of carbazolyl, arylamino, alkylamino, silyl, alkoxy, aryloxy, thio, alkylthio, arylthio, acridinyl, phenoxazine, or phenothiazine;
L is selected from at least one of a single bond, —O—, phenyl, biphenyl, cycloalkylene, arylene, heteroaryl, heterocycloalkylene, or heteroalkenylene, and n is 1-4; and
A is selected from at least one of fluorine, cyano, triazine, cyanobenzene, pyridine, phosphinoxy, ketocarbonyl, sulfonyl, pyrrolyl, thienyl, pyrazolyl, thiazolyl, pyridyl, pyrazinyl, pyrimidinyl, pyridazinyl, or phenalenylene.

In some embodiments, in the above thermally activated delayed fluorescence material provided by the embodiment of the disclosure, the above formula (1) may be selected from the following compounds:

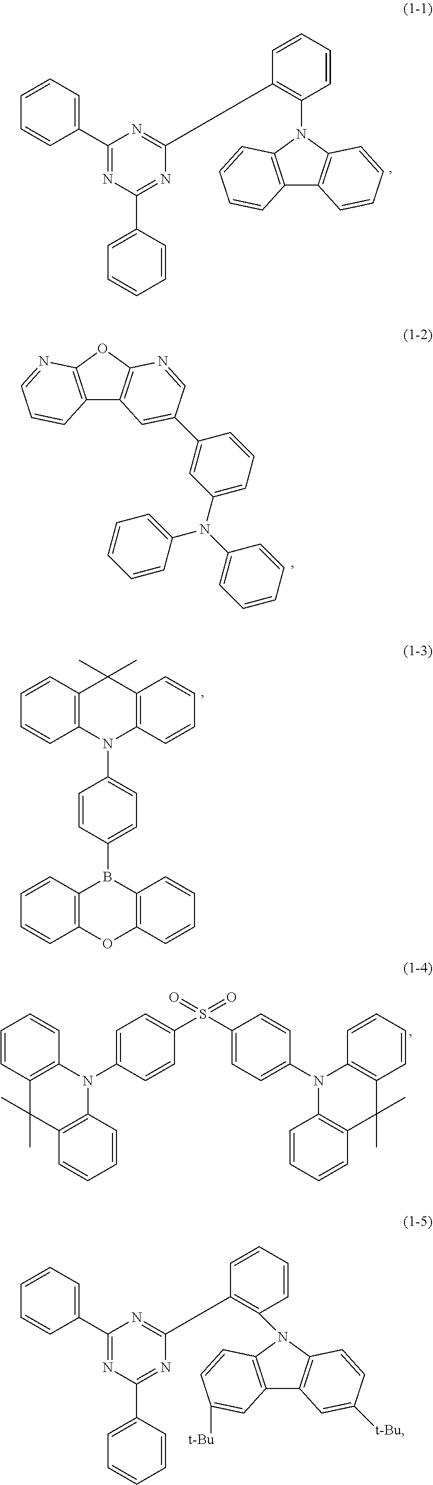 [SMILES: c1ccc(-c2nc(-c3ccccc3)nc(-c3ccccc3-n3c4ccccc4c4ccccc43)n2)cc1 ; c1ccc(N(c2ccccc2)c2cccc(-c3cnc4oc5ncccc5c4c3)c2)cc1 ; CC1(C)c2ccccc2N(c2ccc(B3c4ccccc4Oc4ccccc43)cc2)c2ccccc21 ; CC1(C)c2ccccc2N(c2ccc(S(=O)(=O)c3ccc(N4c5ccccc5C(C)(C)c5ccccc54)cc3)cc2)c2ccccc21 ; CC(C)(C)c1ccc2c(c1)c1cc(C(C)(C)C)ccc1n2-c1ccccc1-c1nc(-c2ccccc2)nc(-c2ccccc2)n1]

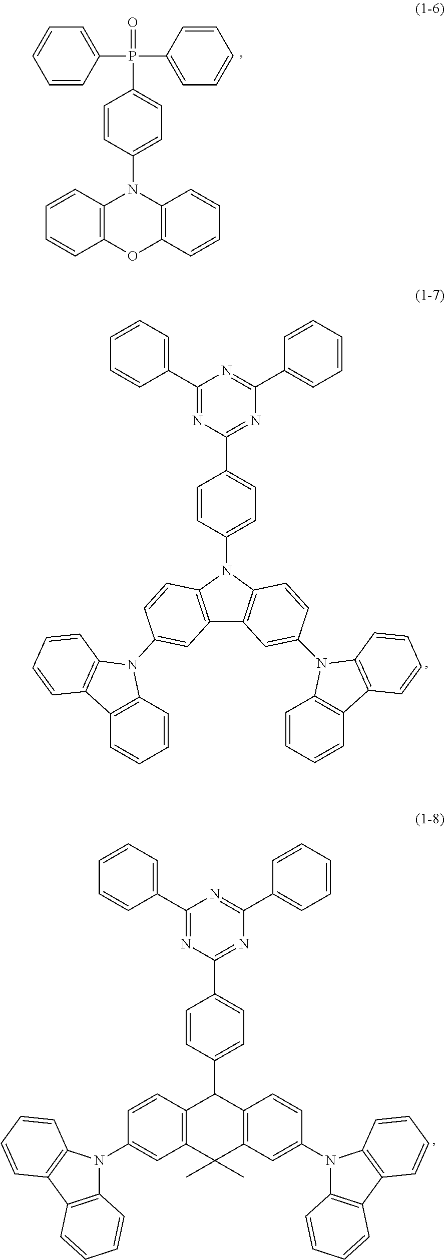 [SMILES: O=P(c1ccccc1)(c1ccccc1)c1ccc(N2c3ccccc3Oc3ccccc32)cc1 ; c1ccc(-c2nc(-c3ccccc3)nc(-c3ccc(-n4c5ccc(-n6c7ccccc7c7ccccc76)cc5c5cc(-n6c7ccccc7c7ccccc76)ccc54)cc3)n2)cc1 ; CC1(C)c2cc(-n3c4ccccc4c4ccccc43)ccc2C(c2ccc(-c3nc(-c4ccccc4)nc(-c4ccccc4)n3)cc2)c2ccc(-n3c4ccccc4c4ccccc43)cc21]

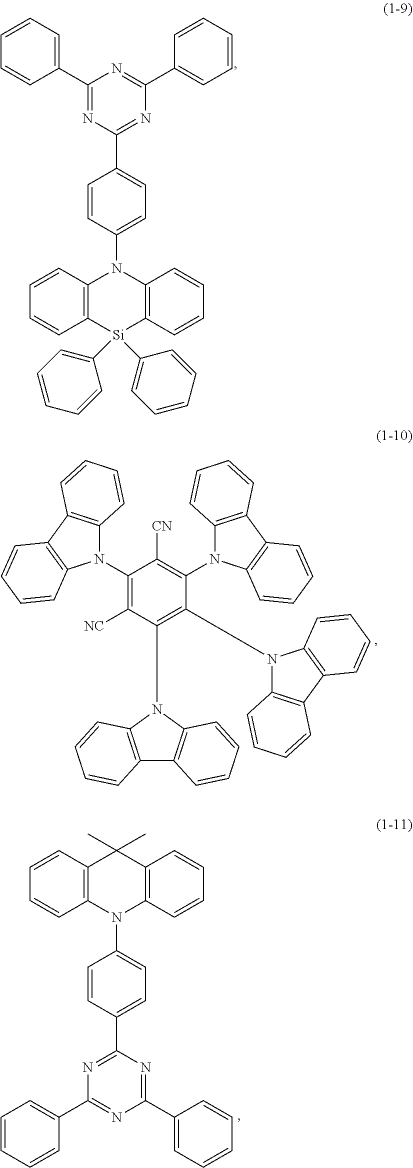 [SMILES: c1ccc(-c2nc(-c3ccccc3)nc(-c3ccc(N4c5ccccc5[Si](c5ccccc5)(c5ccccc5)c5ccccc54)cc3)n2)cc1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21 ; CC1(C)c2ccccc2N(c2ccc(-c3nc(-c4ccccc4)nc(-c4ccccc4)n3)cc2)c2ccccc21]

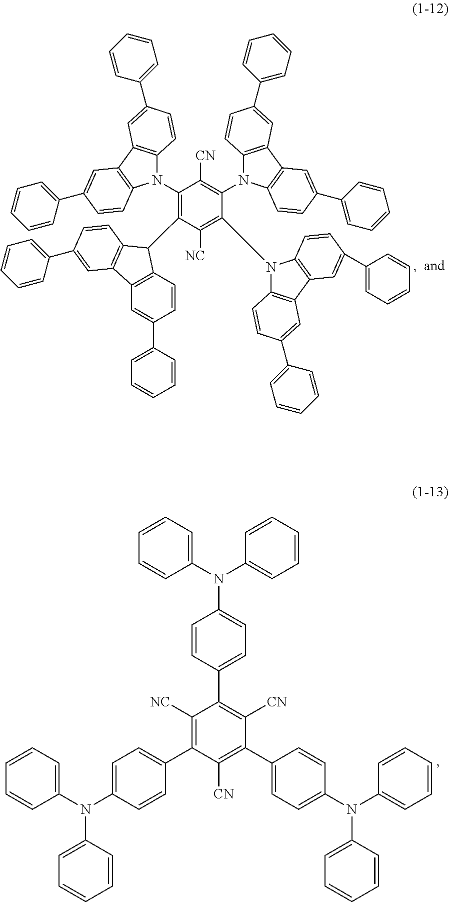 [SMILES: N#Cc1c(C2c3ccc(-c4ccccc4)cc3-c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-c2ccc(N(c3ccccc3)c3ccccc3)cc2)c(C#N)c(-c2ccc(N(c3ccccc3)c3ccccc3)cc2)c(C#N)c1-c1ccc(N(c2ccccc2)c2ccccc2)cc1]

and of course, is not limited thereto.

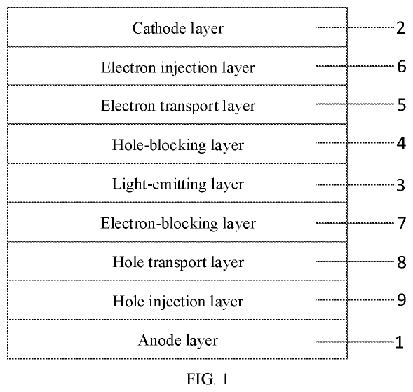

Based on the same inventive concept, an embodiment of the disclosure also provides an organic light-emitting device, as shown in FIG. 1, including:
an anode layer 1;
a cathode layer 2, disposed opposite to the anode layer 1; and
a light-emitting layer 3, between the anode layer 1 and the cathode layer 2, where the light-emitting layer 3 includes a host material, a guest material, and the above thermally activated delayed fluorescence material provided by the embodiment of the disclosure.

According to the above organic light-emitting device provided by the embodiment of the disclosure, a thermally activated delayed fluorescence material having both a large SOC value (≥0.05 cm⁻¹) and small ΔE_{ST} (less than 0.3 eV) is used as a material of the light-emitting layer, which is beneficial to increase the rate of conversion of triplet (T1) excitons to singlet (S1) excitons of the thermally activated delayed fluorescence material when the light-emitting layer emits light, and the triplet excitons in the thermally activated delayed fluorescence material can then more easily conduct reverse intersystem crossing to the singlet state to form singlet excitons, which can greatly reduce the degradation of device performance due to triplet exciton annihilation, improving the device efficiency and lifetime.

With the continuous development of the OLED technology, superfluorescent OLED devices have gradually developed into multilayer-structured thin film devices having a plurality of functional layers, more attention has been paid to the research on efficient organic materials and the device performance that affect superfluorescent OLEDs, and an organic light-emitting device of a superfluorescent system with high efficiency and long lifetime is usually the result of an optimized collocation of various organic materials, especially a collocation of a thermally activated delayed fluorescence material and a hole-blocking layer material. Therefore, the above organic light-emitting device provided by the embodiment of the disclosure, as shown in FIG. 1, further includes a hole-blocking layer 4 between the light-emitting layer 3 and the cathode layer 2, where a material of the hole-blocking layer 4 has a structure represented by the following formula (2):

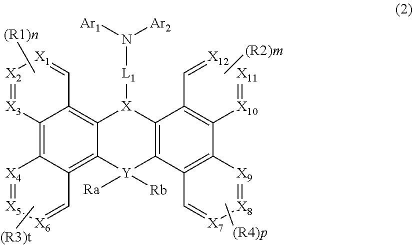

in the formula (2), at least one of X₁ to X₁₂ is N;
X is B or N, Y is C or Si, and n, m, t, and p are each independently an integer from 0-4;
R1 to R4 are each independently substituted or unsubstituted C6-60 aryl, or R1 to R4 are each independently substituted or unsubstituted C2-60 heteroaryl containing heteroatoms selected from any one or more of N, O and S;
L₁ is a single bond, or L₁ is substituted or unsubstituted C6-60 arylene, or L₁ is substituted or unsubstituted C2-60 heteroarylene containing heteroatoms selected from any one or more of N, O, and S; and
Ar₁ and Ar₂ are each independently substituted or unsubstituted C6-60 aryl, or Ar₁ and Ar₂ are each independently substituted or unsubstituted C2-60 heteroaryl containing heteroatoms selected from any one or more of N, O, and S.

The material of the above hole-blocking layer provided by the embodiment of the disclosure, as a hole-blocking layer, has a wide band gap, a high triplet energy level, and high mobility, and can enhance the blocking of exciton diffusion by an electron transport layer (described later), and increase the recombination and use efficiency of carriers, and functions to reduce the device voltage and increase the device efficiency.

In some embodiments, in the above organic light-emitting device provided by the embodiment of the disclosure, the above formula (2) may be selected from the following compounds:

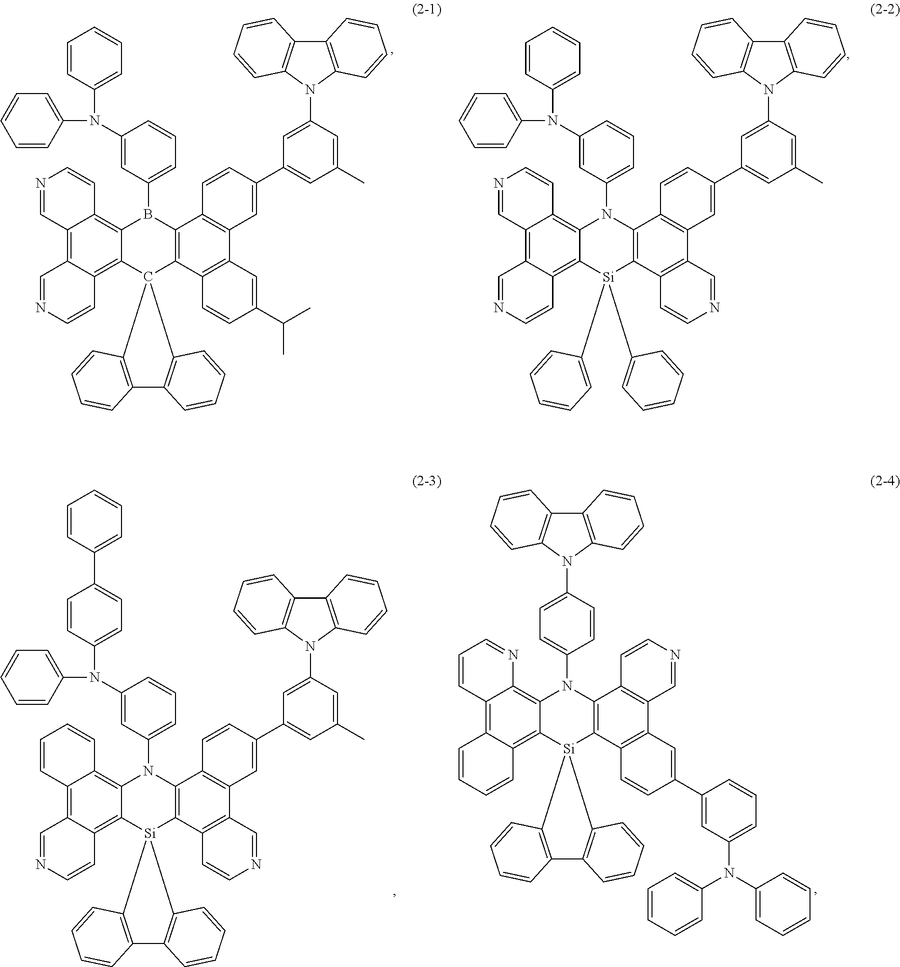

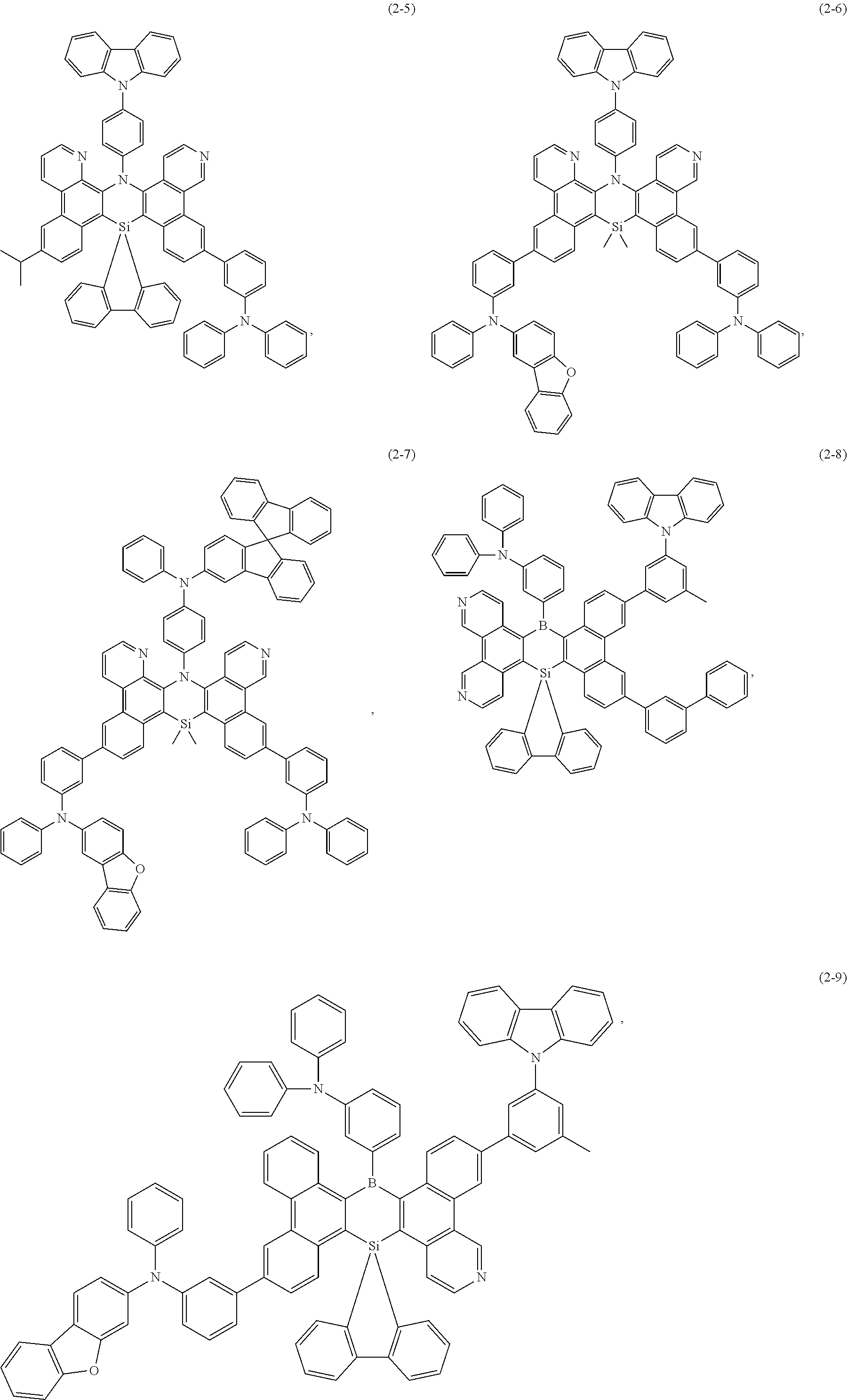

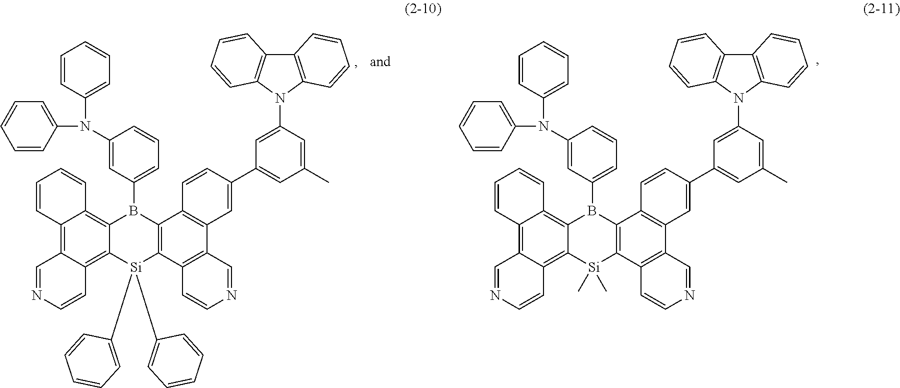 [SMILES: Cc1cc(-c2ccc3c4c(c5ccncc5c3c2)[Si](c2ccccc2)(c2ccccc2)c2c(c3ccccc3c3cnccc23)B4c2cccc(N(c3ccccc3)c3ccccc3)c2)cc(-n2c3ccccc3c3ccccc32)c1 ; Cc1cc(-c2ccc3c4c(c5ccncc5c3c2)[Si](C)(C)c2c(c3ccccc3c3cnccc23)B4c2cccc(N(c3ccccc3)c3ccccc3)c2)cc(-n2c3ccccc3c3ccccc32)c1]

but is not limited thereto.

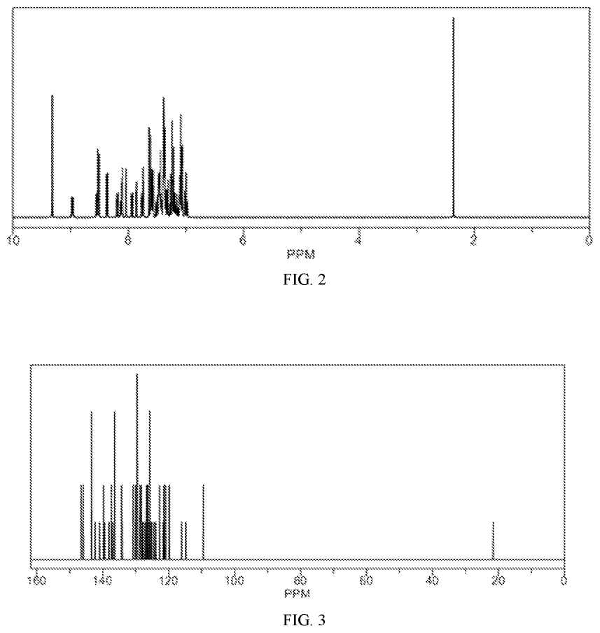

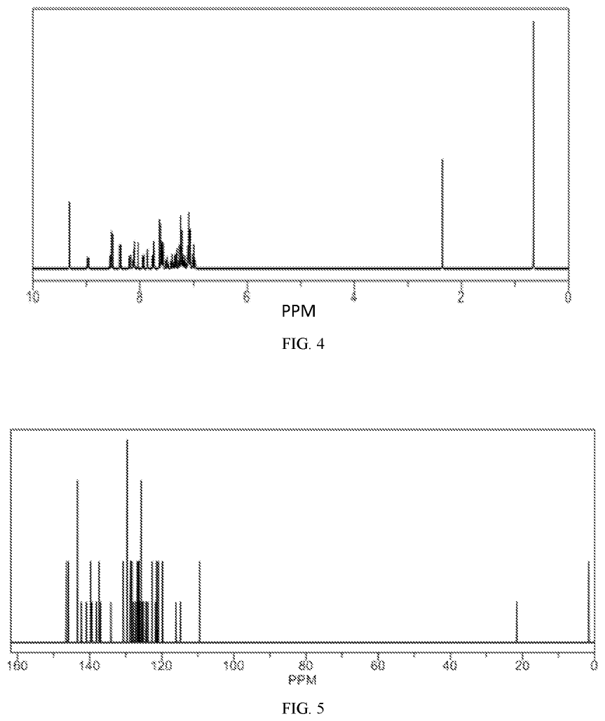

In the embodiments of the disclosure, the hydrogen nuclear magnetic resonance spectrum (1H-NMR) and the carbon nuclear magnetic resonance spectrum (13C-NMR) of the formulas 2-10 and 2-11 above are tested, as shown in FIGS. 2-5, FIG. 2 is a hydrogen nuclear magnetic resonance spectrum (1H-NMR) of the formula 2-10, FIG. 3 is a carbon nuclear magnetic resonance spectrum (13C-NMR) of the formula 2-10, FIG. 4 is a hydrogen nuclear magnetic resonance spectrum (1H-NMR) of the formula 2-11, and FIG. 5 is a carbon nuclear magnetic resonance spectrum (13C-NMR) of the formula 2-11.

In some embodiments, in order to increase the efficiency of the organic light-emitting device, in the above organic light-emitting device provided by the embodiment of the disclosure, as shown in FIG. 1, the lowest triplet energy of the host material in the light-emitting layer 3 is less than the lowest triplet energy of the material of the hole-blocking layer 4, which can prevent the energy of the host material in the light-emitting layer 3 from transferring to the hole-blocking layer 4, further improving the luminous efficiency of the organic light-emitting device.

In some embodiments, in the above organic light-emitting device provided by the embodiment of the disclosure, as shown in FIG. 1, the HOMO energy level of the host material in the light-emitting layer 3 is lower than the HOMO energy level of the material of the hole-blocking layer 4, which can better limit holes in the light-emitting layer 3, prevent the energy of the material of the light-emitting layer 3 from diffusing to peripheral functional layers, and further improve the luminous efficiency of the organic light-emitting device.

In some embodiments, in order to increase the efficiency of the organic light-emitting device, in the above organic light-emitting device provided by the embodiment of the disclosure, as shown in FIG. 1, the lowest triplet energy of the thermally activated delayed fluorescence material in the light-emitting layer 3 is less than the lowest triplet energy of the material of the hole-blocking layer 4, which is conducive to limiting excitons in the light-emitting layer 3, further improving the luminous efficiency of the organic light-emitting device.

In some embodiments, in the above organic light-emitting device provided by the embodiment of the disclosure, as shown in FIG. 1, the HOMO energy level of the thermally activated delayed fluorescence material in the light-emitting layer 3 is lower than the HOMO energy level of the material of the hole-blocking layer 4, which can better limit holes in the thermally activated delayed fluorescence material, prevent the energy of the light-emitting layer 3 from diffusing to peripheral functional layers, and further improve the luminous efficiency of the organic light-emitting device.

It should be noted that the term HOMO stands for “highest occupied molecular orbital” while the term LUMO stands for “lowest unoccupied molecular orbital”.

In some embodiments, the above organic light-emitting device provided by the embodiment of the disclosure, as shown in FIG. 1, further includes an electron transport layer 5 between the hole-blocking layer 4 and the cathode layer 2, an electron injection layer 6 between the electron transport layer 5 and the cathode layer 2, an electron-blocking layer 7 between the light-emitting layer 3 and the anode layer 1, a hole transport layer 8 between the electron-blocking layer 7 and the anode layer 1, and a hole injection layer 9 between the hole transport layer 8 and the anode layer.

In particular, a material of the hole injection layer may be an inorganic oxide, such as molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, and the like, and may also be a doped substance with a p-type dopant of a strong electron withdrawing system and a hole transport material, such as hexacyanohexaazatriphenylene,
2,3,5,6-tetrafluoro-7,7, 8,8-tetracyano-p-quinodimethane (F4TCNQ),
1,2,3-tris[(cyano)(4-cyano-2,3,5,6-tetrafluorophenyl)methylene]cyclopropane, and the like.

In particular, a material of the hole transport layer/the electron-blocking layer may be an arylamine or carbazole material having hole transport properties, such as
4,4′-bis[N-(1-naphthyl)-N-phenylamino]biphenyl (NPB),
N,N′-bis(3-methylphenyl)-N,N′-diphenyl-[1,1′-biphenyl]-4,4′-diamine (TPD),
4-phenyl-4′-(9-phenylfluoren-9-yl)triphenylamine (BAFLP),
4,4′-bis[N-(9,9-dimethylfluoren-2-yl)-N-phenylamino]biphenyl (DFLDPBi),
4,4′-di(9-carbazolyl)biphenyl (CBP), 9-phenyl-3-[4-(10-phenyl-9-anthryl)phenyl]-9H-carbazole (PCzPA), and the like.

In particular, a material of the light-emitting layer includes three compounds, and may include a metal complex. The light-emitting layer is preferably free of a phosphorescent light-emitting metal complex.

Specifically, the host material (also referred to as a matrix material) in the light-emitting layer, for example, includes a hole-type material containing carbazole, spirofluorene, or a biphenyl group; and the guest material (also referred to as a luminescent substance or a light-emitting material) can be a fluorescent material or a phosphorescent material, and the guest material is preferably a fluorescent light-emitting material.

Specifically, the electron transport layer is generally made of an aromatic heterocyclic compound, such as an imidazole derivative such as a benzimidazole derivative, an imidazopyridine derivative, and a benzimidazophenanthridine derivative; an azine derivative such as a pyrimidine derivative and a triazine derivative; a compound containing a nitrogen-containing six-membered ring structure such as a quinoline derivative, an isoquinoline derivative, and a phenanthroline derivative (also including a compound having a phosphine oxide-based substituent on a heterocyclic ring) and the like, specifically, for example,
2-(4-biphenyl)-5-(4-tert-butylphenyl)-1,3,4-oxadiazole (PBD),
1,3-bis[5-(p-tert-butylphenyl)-1,3,4-oxadiazol-2-yl]benzene (OXD-7),
3-(4-tert-butylphenyl)-4-phenyl-5-(4-biphenyl)-1,2,4-triazole (TAZ),
3-(4-tert-butylphenyl)-4-(4-ethylphenyl)-5-(4-biphenyl)-1,2,4-triazole (p-EtTAZ),
bathophenanthroline (BPhen), bathocuproine (BCP), 4,4′-bis(5-methylbenzoxazol-2-yl)stilbene (BzOs), and the like.

In particular, the electron injection layer is generally made of an alkali metal or metal, such as LiF, Yb, Mg, Ca, or compounds thereof, etc.

Specifically, 13 thermally activated delayed fluorescence materials provided by the embodiment of the disclosure are respectively labeled 1-1 to 1-13, and an embodiment of the disclosure provides the SOC values and ΔE_{S1T1} of the first nine thermally activated delayed fluorescence materials, as shown in Table (1) below.

TABLE 1

| Material | SOC value (cm⁻¹) | ΔE_{S1T1} (eV) |
|---|---|---|
| 1-1 | 0.18 | 0.04 |
| 1-2 | 0.13 | 0.16 |
| 1-3 | 0.14 | 0.21 |
| 1-4 | 0.62 | 0.16 |
| 1-5 | 0.15 | 0.24 |
| 1-6 | 0.08 | 0.08 |
| 1-7 | 0.09 | 0.30 |
| 1-8 | 0.16 | 0.23 |
| 1-9 | 0.06 | 0.12 |

Taking the structure of the organic light-emitting device shown in FIG. 1 as an example, the device efficiency and service life of the light-emitting layer including the above thermally activated delayed fluorescence material provided by the embodiment of the disclosure are tested, and the parameters of a specific device structure are as follows: the anode layer 1 is made of an ITO material, the hole injection layer 9 is made of a hole-type material containing 2% of a dopant (an organic semiconductor), the hole injection layer 9 has a thickness of 10 nm, the hole transport layer 8 has a thickness of 195 nm, the electron-blocking layer 7 has a thickness of 5 nm, a ratio of the host material to the thermally activated delayed fluorescence material to the guest material in the light-emitting layer 3 is 69%:30%:1%, the light-emitting layer 3 has a thickness of 25 nm, the hole-blocking layer 4 has a thickness of 5 nm, the electron transport layer 5 has a thickness of 30 nm, the electron injection layer 6 has a thickness of 0.5 nm, the cathode layer 2 is made of a Mg/Ag alloy, a ratio of Mg to Ag is 9:1, and the cathode layer 2 has a thickness of 130 nm.

The material structures of the film layers of the organic light-emitting device shown in FIG. 1 are as follows: HIL stands for hole injection layer, HTL stands for hole transport layer, EBL stands for electron-blocking layer, Host represents the host material of the light-emitting layer, Dopant represents the guest material of the light-emitting layer, ETL stands for electron transport layer, and LiQ represents the electron injection layer.

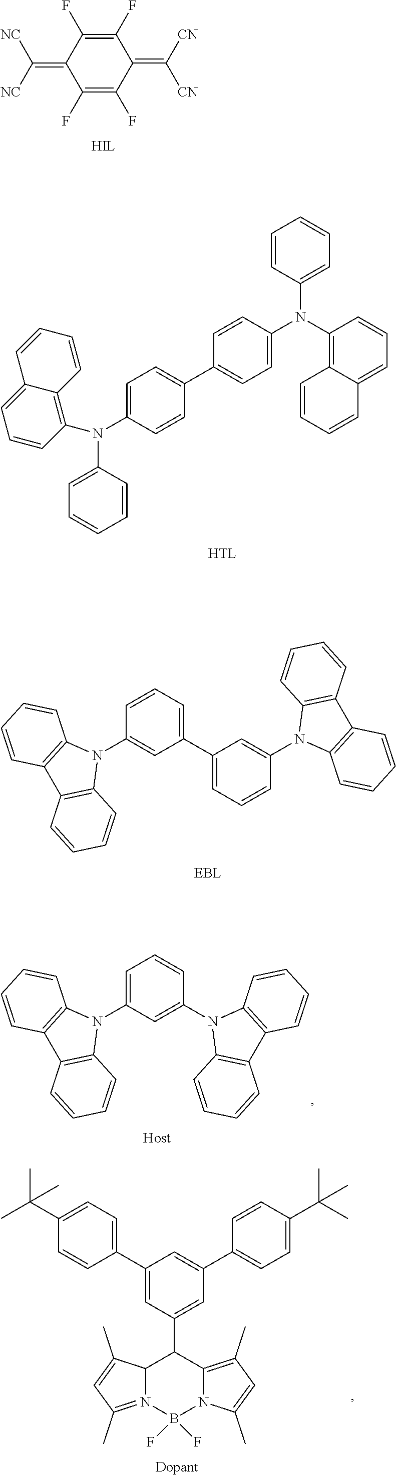

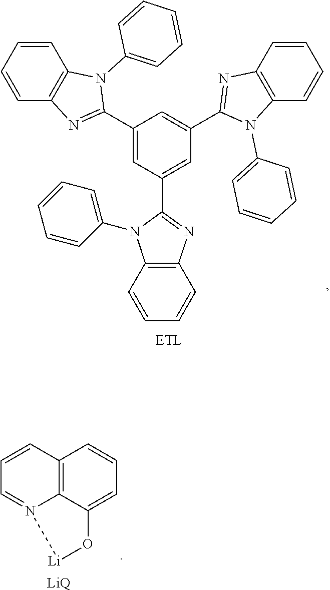

In the embodiments of the disclosure, the device service life and chromaticity coordinates in the six embodiments with six combinations of thermally activated delayed fluorescence materials and hole-blocking layer materials are obtained as shown in Table (2).

TABLE (2)

| | | | | | | Service life |
|---|---|---|---|---|---|---|
| | | | | | | (LT95@1000 |
| Material combination | | Voltage | Efficiency | CIEx | CIEy | nit) |
| Embodiment 1 | 2-11 | 4.82 | 43.52 | 0.332 | 0.645 | 100% |
| | and 1-1 | | | | | |
| Embodiment 2 | 2-10 | 4.73 | 41.45 | 0.327 | 0.647 | 101% |
| | and 1-2 | | | | | |
| Embodiment 3 | 2-6 and | 4.68 | 40.41 | 0.340 | 0.639 | 94% |
| | 1-4 | | | | | |
| Embodiment 4 | 2-4 and | 4.48 | 36.73 | 0.337 | 0.640 | 103% |
| | 1-1 | | | | | |
| Embodiment 5 | 2-7 and | 4.34 | 33.22 | 0.348 | 0.631 | 73% |
| | 1-2 | | | | | |
| Embodiment 6 | 2-1 and | 5.28 | 30.2 | 0.362 | 0.618 | 82% |
| | 1-4 | | | | | |

As can be seen from the above Table (2), the efficiency and service life of the organic light-emitting device are higher by using the energy level matching and material combinations according to the embodiments of the disclosure.

Based on the same inventive concept, an embodiment of the disclosure also provides a display apparatus, including the organic light-emitting device in the above embodiment. Since the principle of solving the problem of the display apparatus is similar to that of the above organic light-emitting device, implementation of the display apparatus may refer to the implementation of the above organic light-emitting device, and repetitions are omitted.

The display apparatus provided by the embodiment of the disclosure may be a mobile phone, a tablet PC, a TV, a display, a notebook computer, a digital photo frame, a navigator or any other product or component with a display function. Other essential components of the display apparatus should be understood by those of ordinary skill in the art, and will not be repeated here, nor should they be regarded as a limitation to the disclosure.

According to the thermally activated delayed fluorescence material, the organic light-emitting device, and the display apparatus provided by the embodiments of the disclosure, a thermally activated delayed fluorescence material having both a large SOC value (>0.05 cm⁻¹) and small ΔE_{ST} (less than 0.3 eV) is used, which is beneficial to increase the rate of conversion of triplet (T1) excitons to singlet (S1) excitons of the thermally activated delayed fluorescence material, the triplet excitons in the thermally activated delayed fluorescence material can then more easily conduct reverse intersystem crossing to the singlet state to form singlet excitons, and when the thermally activated delayed fluorescence material is applied to the light-emitting layer of the organic light-emitting device, the reduction in device performance due to triplet exciton annihilation can be greatly reduced.

Obviously, those skilled in the art can make various modifications and variations on the disclosure without departing from the spirit and scope of the disclosure. Thus, if these modifications and variations of the disclosure belong to the scope of the claims of the disclosure and their equivalents, the disclosure is also intended to contain these modifications and variations.

1. A thermally activated delayed fluorescence material, wherein
an energy gap between a singlet state and a triplet state of the thermally activated delayed fluorescence material is less than 0.3 eV; and
a spin-orbit coupling (SOC) value between the singlet state and the triplet state of the thermally activated delayed fluorescence material is greater than or equal to 0.05 cm⁻³.

2. The thermally activated delayed fluorescence material according to claim 1, wherein the thermally activated delayed fluorescence material has a structure represented by formula (1):
D-Ln-A (1)
in the formula (1), D is a donor group, L is a linking group, and A is an acceptor group;
wherein D is selected from at least one of carbazolyl, arylamino, alkylamino, silyl, alkoxy, aryloxy, thio, alkylthio, arylthio, acridinyl, phenoxazine, or phenothiazine;
L is selected from at least one of a single bond, —O—, phenyl, biphenyl, cycloalkylene, arylene, heteroaryl, heterocycloalkylene, or heteroalkenylene, and n is 1-4; and
A is selected from at least one of fluorine, cyano, triazine, cyanobenzene, pyridine, phosphinoxy, ketocarbonyl, sulfonyl, pyrrolyl, thienyl, pyrazolyl, thiazolyl, pyridyl, pyrazinyl, pyrimidinyl, pyridazinyl, or phenalenylene.

3. The thermally activated delayed fluorescence material according to claim 2, wherein the formula (1) is selected from following compounds:

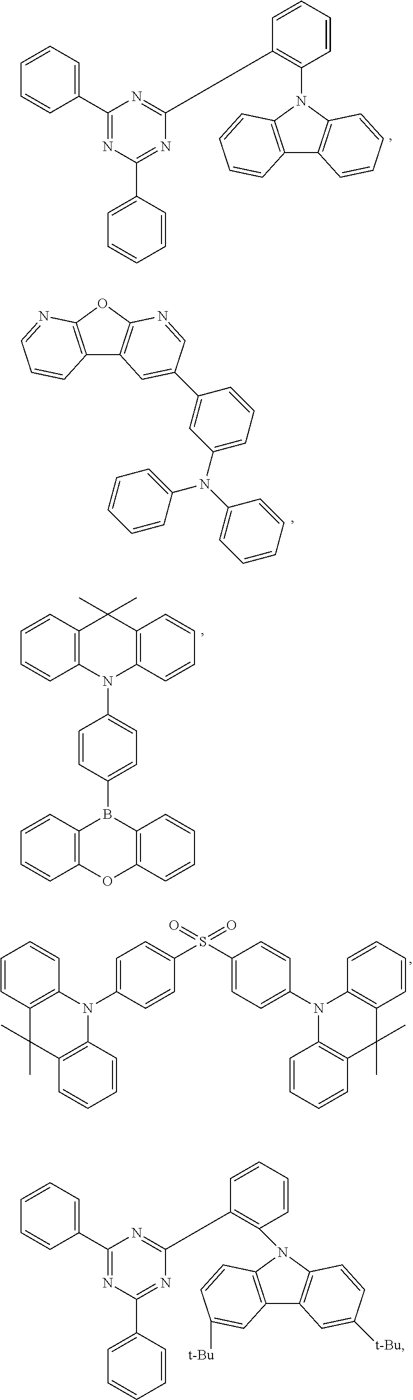

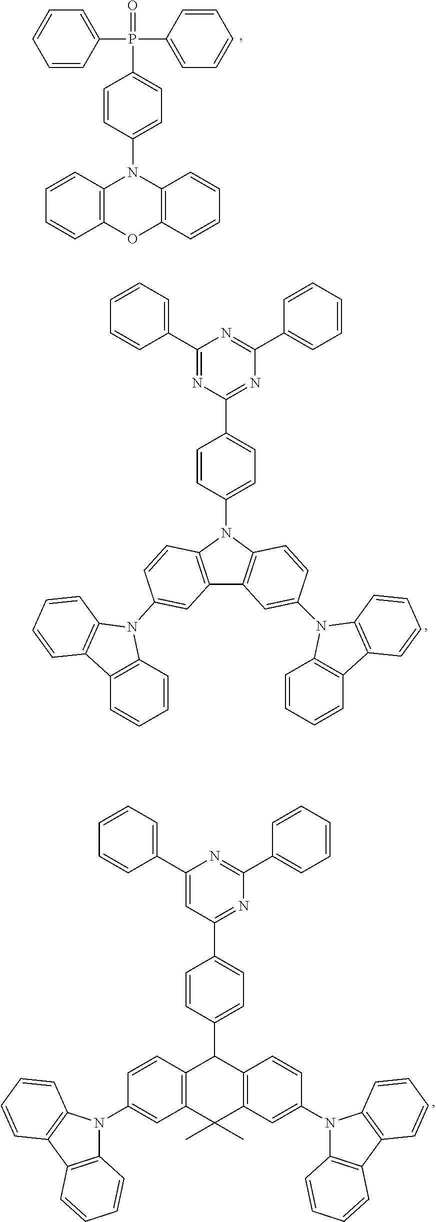

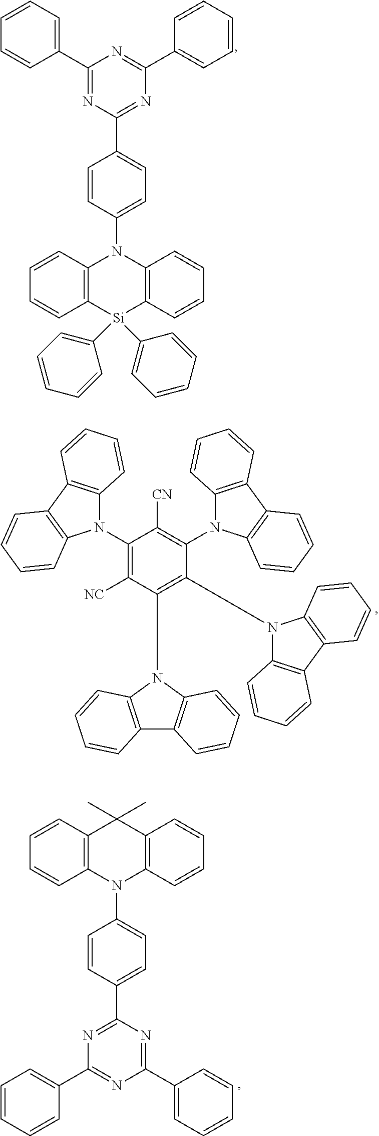

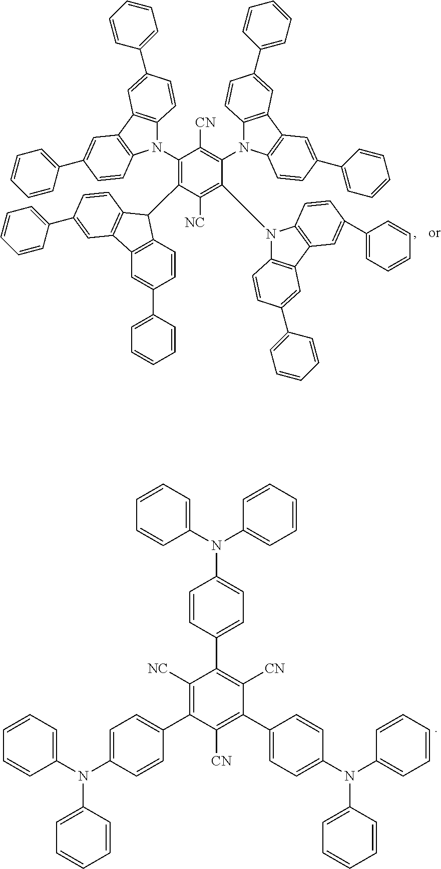 [SMILES: N#Cc1c(C2c3ccc(-c4ccccc4)cc3-c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-c2ccc(N(c3ccccc3)c3ccccc3)cc2)c(C#N)c(-c2ccc(N(c3ccccc3)c3ccccc3)cc2)c(C#N)c1-c1ccc(N(c2ccccc2)c2ccccc2)cc1]

4. An organic light-emitting device, comprising:
an anode layer;
a cathode layer, disposed opposite to the anode layer; and
a light-emitting layer, disposed between the anode layer and the cathode layer;
wherein the light-emitting layer comprises a host material, a guest material, and a thermally activated delayed fluorescence material, wherein
an energy gap between a singlet state and a triplet state of the thermally activated delayed fluorescence material is less than 0.3 eV; and
a spin-orbit coupling (SOC) value between the singlet state and the triplet state of the thermally activated delayed fluorescence material is greater than or equal to 0.05 cm⁻³.

5. The organic light-emitting device according to claim 4, further comprising a hole-blocking layer between the light-emitting layer and the cathode layer, wherein a material of the hole-blocking layer has a structure represented by formula (2): 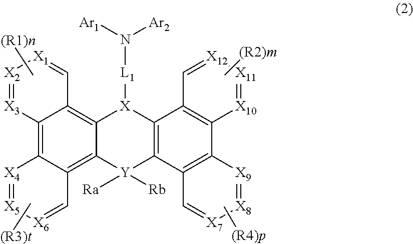
in the formula (2), at least one of X₁ to X₁₂ is N;
X is B or N, Y is C or Si, and n, m, t, and p are each independently an integer from 0-4;
R1 to R4 are each independently substituted or unsubstituted C6-60 aryl, or R1 to R4 are each independently substituted or unsubstituted C2-60 heteroaryl containing heteroatoms selected from any one or more of N, O and S;
L₁ is a single bond, or L₁ is substituted or unsubstituted C6-60 arylene, or L₁ is substituted or unsubstituted C2-60 heteroarylene containing heteroatoms selected from any one or more of N, O, and S; and
Ar₁ and Ar₂ are each independently substituted or unsubstituted C6-60 aryl, or Ar₁ and Ar₂ are each independently substituted or unsubstituted C2-60 heteroaryl containing heteroatoms selected from any one or more of N, O, and S.

6. The organic light-emitting device according to claim 5, wherein the formula (2) is selected from following compounds:

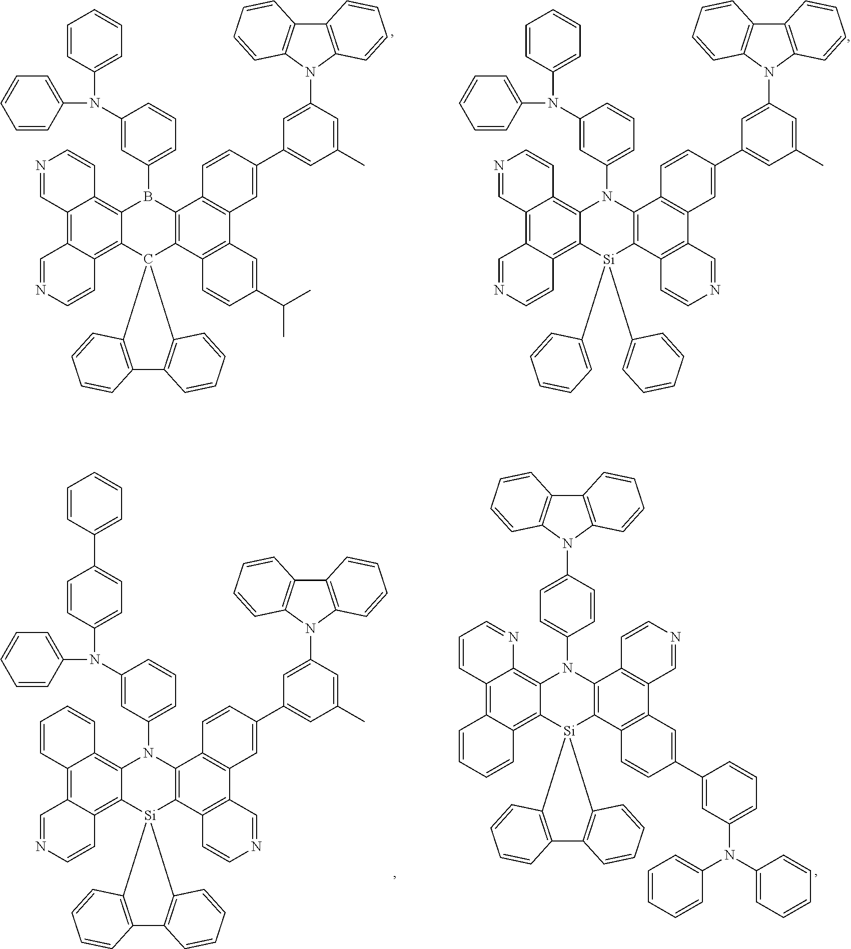

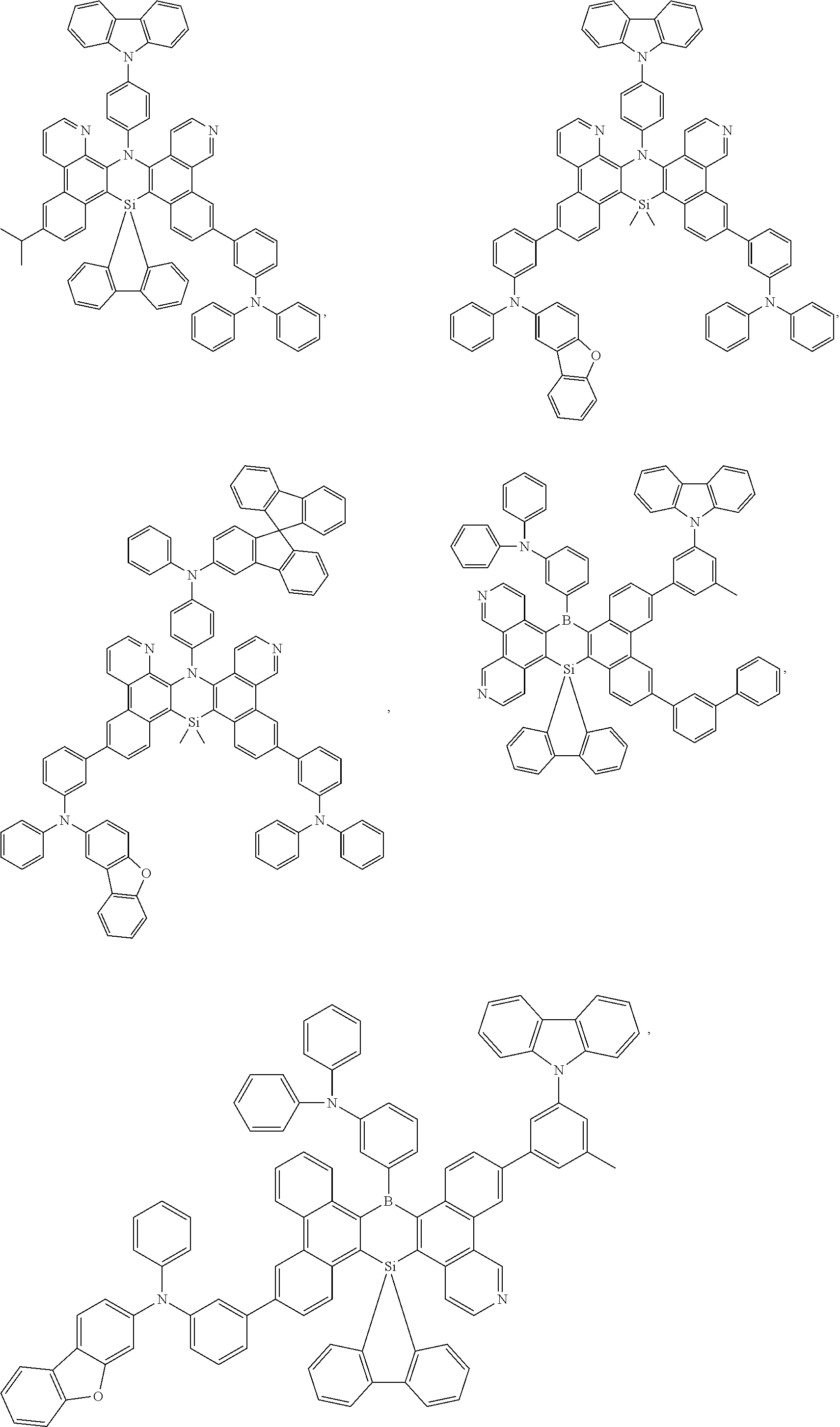

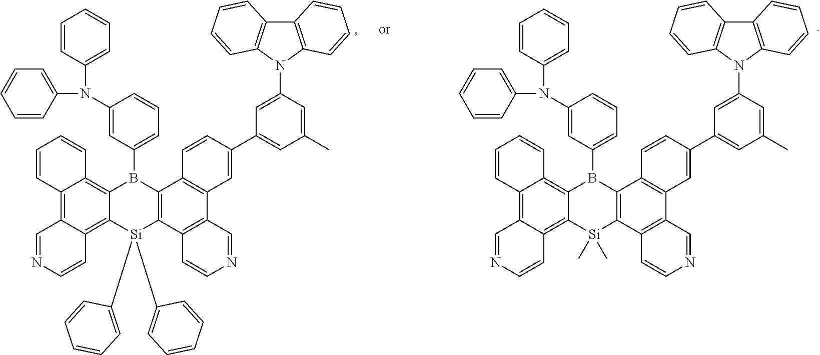 [SMILES: Cc1cc(-c2ccc3c4c(c5ccncc5c3c2)[Si](c2ccccc2)(c2ccccc2)c2c(c3ccccc3c3cnccc23)B4c2cccc(N(c3ccccc3)c3ccccc3)c2)cc(-n2c3ccccc3c3ccccc32)c1 ; Cc1cc(-c2ccc3c4c(c5ccncc5c3c2)[Si](C)(C)c2c(c3ccccc3c3cnccc23)B4c2cccc(N(c3ccccc3)c3ccccc3)c2)cc(-n2c3ccccc3c3ccccc32)c1]

7. The organic light-emitting device according to claim 5, wherein energy of a lowest triplet state of the host material in the light-emitting layer is lower than energy of a lowest triplet state of the material of the hole-blocking layer.

8. The organic light-emitting device according to claim 5, wherein a HOMO energy level of the host material in the light-emitting layer is lower than a HOMO energy level of the material of the hole-blocking layer.

9. The organic light-emitting device according to claim 5, wherein energy of a lowest triplet state of the thermally activated delayed fluorescence material in the light-emitting layer is lower than energy of a lowest triplet state of the material of the hole-blocking layer.

10. The organic light-emitting device according to claim 5, wherein a HOMO energy level of the thermally activated delayed fluorescence material in the light-emitting layer is less than a HOMO energy level of the material of the hole-blocking layer.

11. The organic light-emitting device according to claim 5, further comprising:
an electron transport layer between the hole-blocking layer and the cathode layer;
an electron injection layer between the electron transport layer and the cathode layer;
an electron-blocking layer between the light-emitting layer and the anode layer;
a hole transport layer between the electron-blocking layer and the anode layer; and
a hole injection layer between the hole transport layer and the anode layer.

12. The organic light-emitting device according to claim 4, wherein the guest material is a fluorescent material or a phosphorescent material.

13. The organic light-emitting device according to claim 11, wherein
a material of the anode layer is ITO;
a material of the hole injection layer is 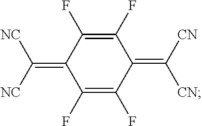 [SMILES: N#CC(C#N)=c1c(F)c(F)c(=C(C#N)C#N)c(F)c1F]
a material of the hole transport layer is 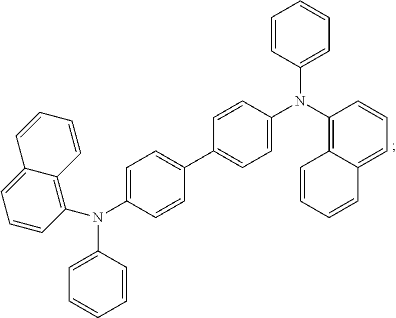 [SMILES: c1ccc(N(c2ccc(-c3ccc(N(c4ccccc4)c4cccc5ccccc45)cc3)cc2)c2cccc3ccccc23)cc1]
a material of the electron-blocking layer is 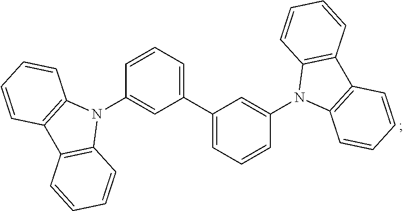 [SMILES: c1cc(-c2cccc(-n3c4ccccc4c4ccccc43)c2)cc(-n2c3ccccc3c3ccccc32)c1]
the host material is 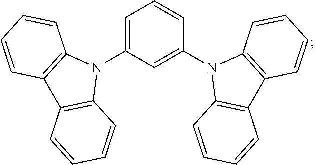 [SMILES: c1cc(-n2c3ccccc3c3ccccc32)cc(-n2c3ccccc3c3ccccc32)c1]
the thermally activated delayed fluorescence material is 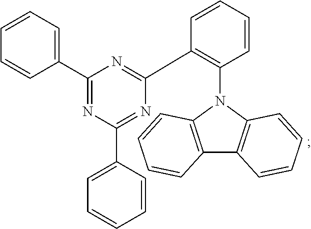 [SMILES: c1ccc(-c2nc(-c3ccccc3)nc(-c3ccccc3-n3c4ccccc4c4ccccc43)n2)cc1]
the guest material is 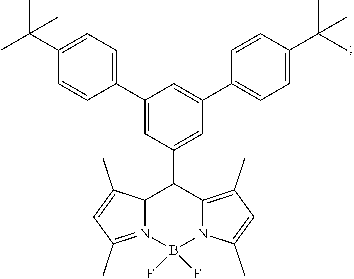
a material of the hole-blocking layer is 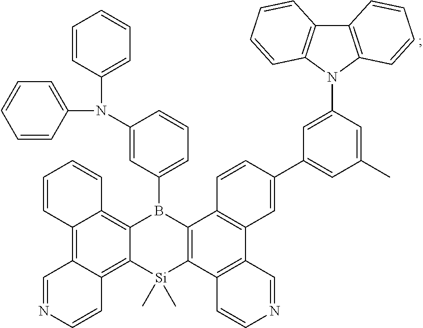 [SMILES: Cc1cc(-c2ccc3c4c(c5ccncc5c3c2)[Si](C)(C)c2c(c3ccccc3c3cnccc23)B4c2cccc(N(c3ccccc3)c3ccccc3)c2)cc(-n2c3ccccc3c3ccccc32)c1]
a material of the electron transport layer is 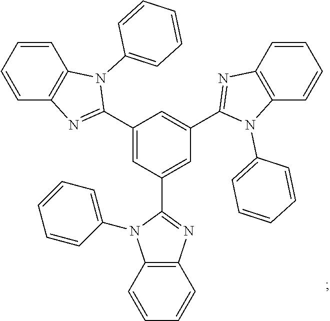 [SMILES: c1ccc(-n2c(-c3cc(-c4nc5ccccc5n4-c4ccccc4)cc(-c4nc5ccccc5n4-c4ccccc4)c3)nc3ccccc32)cc1]
a material of the electron injection layer is 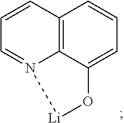
and
a material of the cathode layer is a Mg/Ag alloy.

14. A display apparatus, comprising the organic light-emitting device according to claim 4.

15. The organic light-emitting device according to claim 4, wherein the thermally activated delayed fluorescence material has a structure represented by formula (1):
D-Ln-A (1)
in the formula (1), D is a donor group, L is a linking group, and A is an acceptor group;
wherein D is selected from at least one of carbazolyl, arylamino, alkylamino, silyl, alkoxy, aryloxy, thio, alkylthio, arylthio, acridinyl, phenoxazine, or phenothiazine;
L is selected from at least one of a single bond, —O—, phenyl, biphenyl, cycloalkylene, arylene, heteroaryl, heterocycloalkylene, or heteroalkenylene, and n is 1-4; and
A is selected from at least one of fluorine, cyano, triazine, cyanobenzene, pyridine, phosphinoxy, ketocarbonyl, sulfonyl, pyrrolyl, thienyl, pyrazolyl, thiazolyl, pyridyl, pyrazinyl, pyrimidinyl, pyridazinyl, or phenalenylene.

16. The organic light-emitting device according to claim 15, wherein the formula (1) is selected from following compounds:

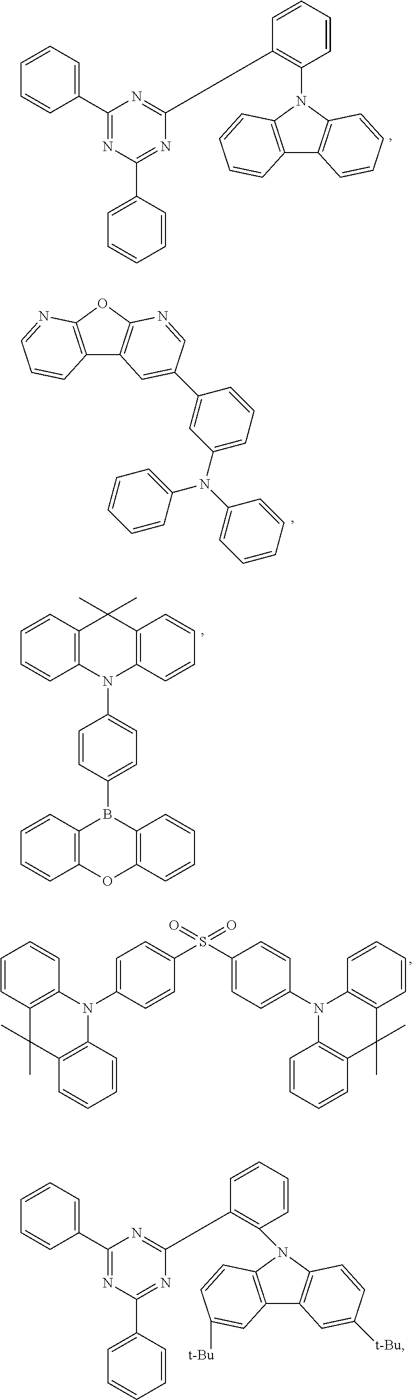

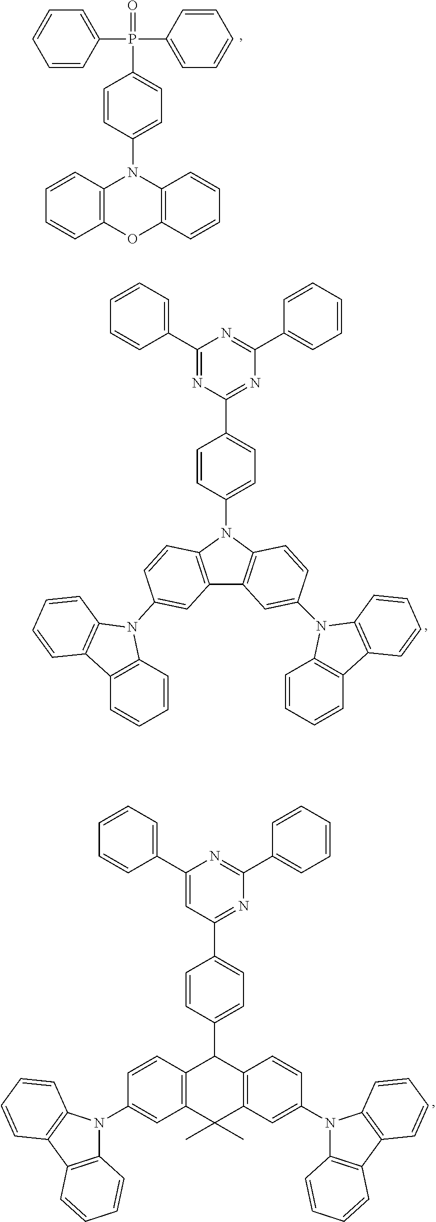

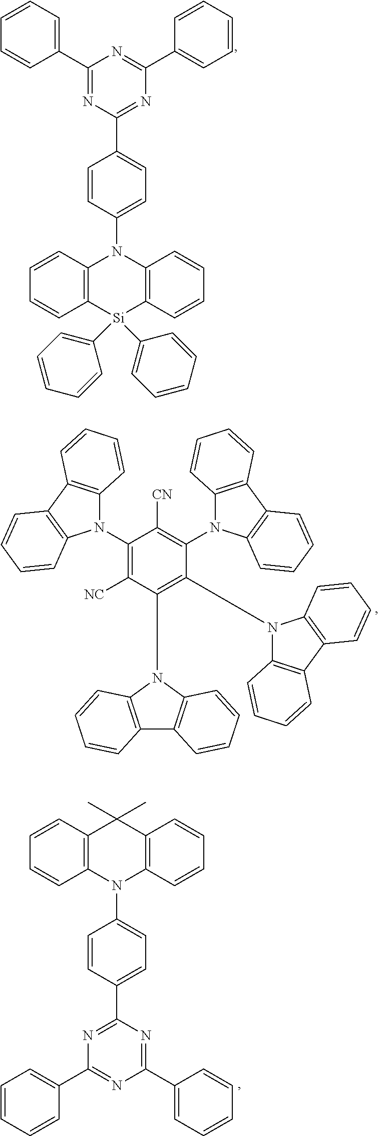

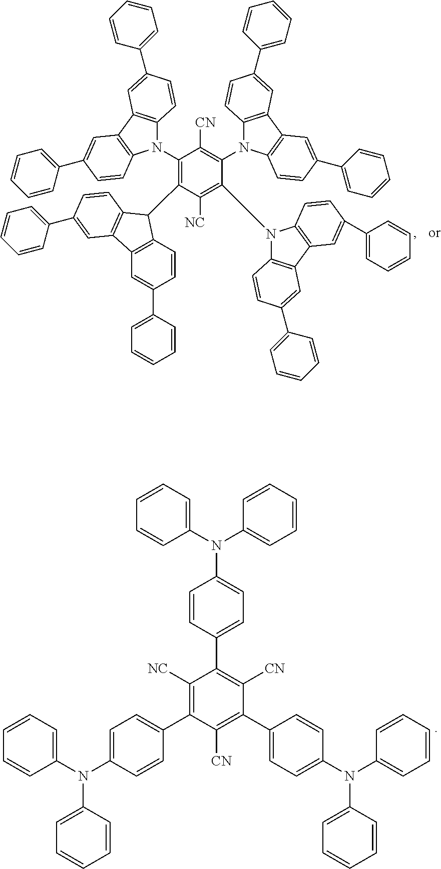 [SMILES: N#Cc1c(C2c3ccc(-c4ccccc4)cc3-c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-c2ccc(N(c3ccccc3)c3ccccc3)cc2)c(C#N)c(-c2ccc(N(c3ccccc3)c3ccccc3)cc2)c(C#N)c1-c1ccc(N(c2ccccc2)c2ccccc2)cc1]

17. The display apparatus according to claim 14, wherein the organic light-emitting device further comprises a hole-blocking layer between the light-emitting layer and the cathode layer, wherein a material of the hole-blocking layer has a structure represented by formula (2): 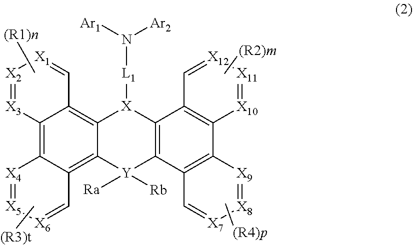
in the formula (2), at least one of X₁ to X₁₂ is N;
X is B or N, Y is C or Si, and n, m, t, and p are each independently an integer from 0-4;
R1 to R4 are each independently substituted or unsubstituted C6-60 aryl, or R1 to R4 are each independently substituted or unsubstituted C2-60 heteroaryl containing heteroatoms selected from any one or more of N, O and S;
L₁ is a single bond, or L₁ is substituted or unsubstituted C6-60 arylene, or L₁ is substituted or unsubstituted C2-60 heteroarylene containing heteroatoms selected from any one or more of N, O, and S; and
Ar₁ and Ar₂ are each independently substituted or unsubstituted C6-60 aryl, or A₁ and Ar₂ are each independently substituted or unsubstituted C2-60 heteroaryl containing heteroatoms selected from any one or more of N, O, and S.

18. The display apparatus according to claim 17, wherein the formula (2) is selected from following compounds:

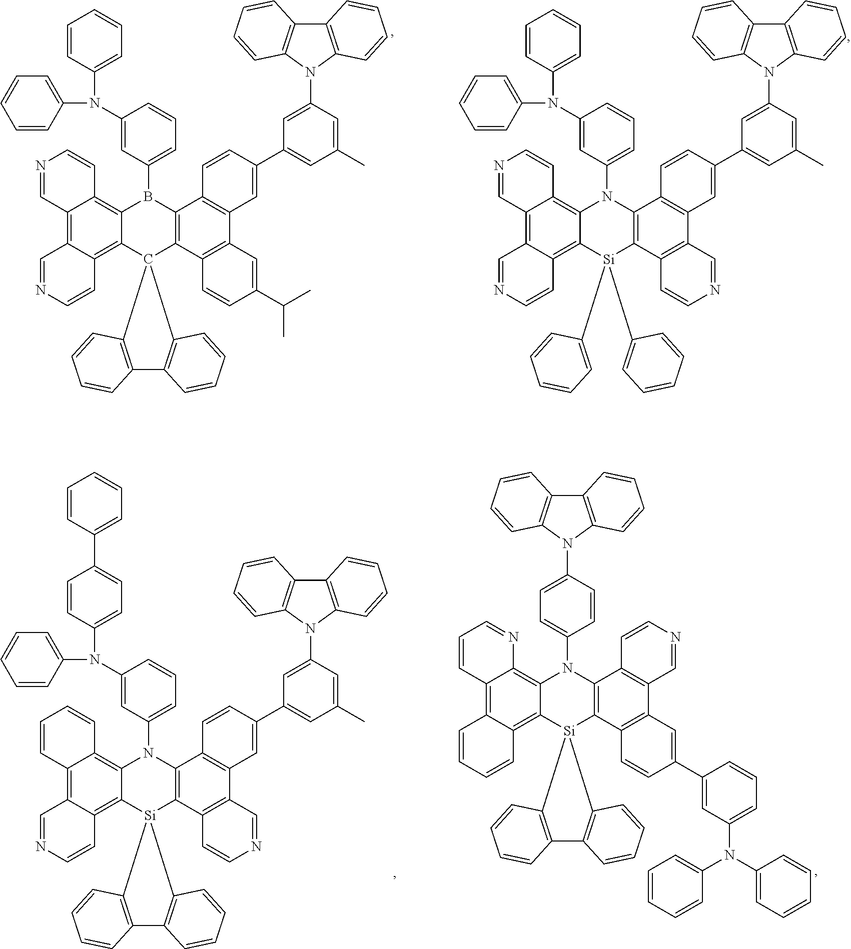

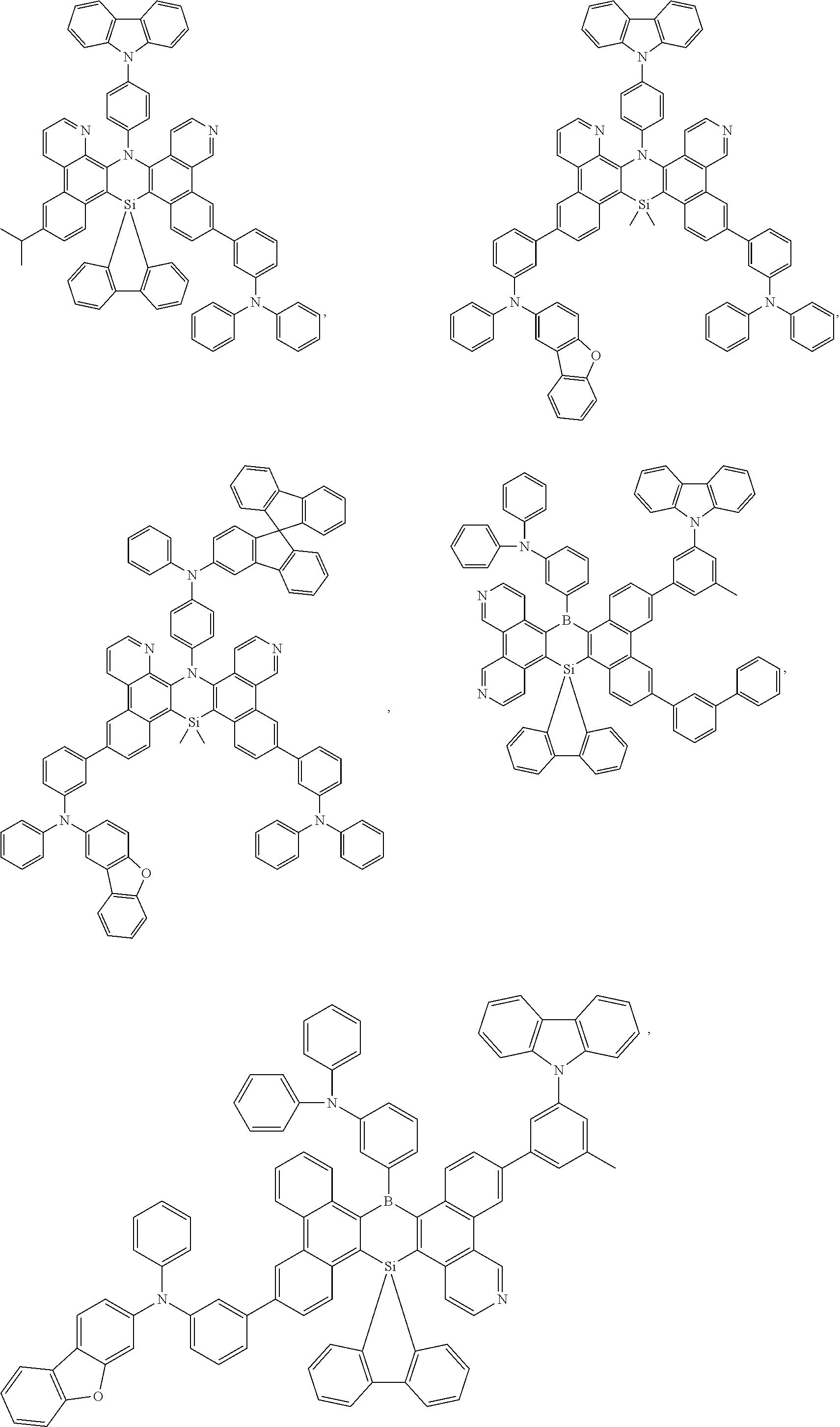

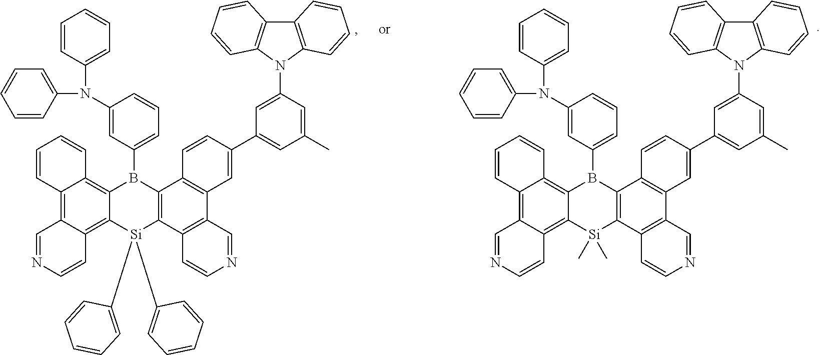 [SMILES: Cc1cc(-c2ccc3c4c(c5ccncc5c3c2)[Si](c2ccccc2)(c2ccccc2)c2c(c3ccccc3c3cnccc23)B4c2cccc(N(c3ccccc3)c3ccccc3)c2)cc(-n2c3ccccc3c3ccccc32)c1 ; Cc1cc(-c2ccc3c4c(c5ccncc5c3c2)[Si](C)(C)c2c(c3ccccc3c3cnccc23)B4c2cccc(N(c3ccccc3)c3ccccc3)c2)cc(-n2c3ccccc3c3ccccc32)c1]

19. The display apparatus according to claim 17, wherein energy of a lowest triplet state of the host material in the light-emitting layer is lower than energy of a lowest triplet state of the material of the hole-blocking layer.

20. The display apparatus according to claim 17, wherein a HOMO energy level of the host material in the light-emitting layer is lower than a HOMO energy level of the material of the hole-blocking layer.
